(19) United States
(12) Patent Application Publication
(10) Pub. No.: US 2024/0417717 A1
(43) Pub. Date: Dec. 19, 2024

(54) METHODS AND NUCLEIC ACID MOLECULES FOR AAV VECTOR SELECTION

(71) Applicant: Children's Medical Research Institute, Westmead (AU)

(72) Inventors: Adrian Westhaus, Westmead (AU); Leszek Lisowski, Westmead (AU)

(21) Appl. No.: 18/701,791

(22) PCT Filed: Oct. 18, 2022
(86) PCT No.: PCT/AU2022/051252

§ 371 Date: Apr. 16, 2024

(30) Foreign Application Priority Data
Oct. 18, 2021 (AU) 2021903328

(51) Int. Cl. C12N 15/10

(52) U.S. Cl. CPC C12N 15/1082

(57) ABSTRACT

Provided herein are methods for identifying novel cap genes suitable for vectorization, utilizing selection based on homologous recombination-mediated editing of a distinct genetic basis. Also provided is the selection and production of AAV vectors that can facilitate homologous recombination-mediated gene editing in a host cell or tissue of choice. Also provided are replication-incompetent AAV and AAV libraries for use in the methods, and nucleic acid molecules to generate the replication-incompetent AAV particles and AAV libraries.

### FIELD OF THE DISCLOSURE

The present disclosure relates generally to nucleic acid molecules and methods for identifying AAV vectors with desirable properties, including nucleic acid molecules and methods useful for identifying novel cap genes suitable for vectorization and subsequent use in gene editing.

### BACKGROUND OF THE DISCLOSURE

Gene therapy and gene editing has been investigated and achieved using viral vectors, with notable recent advances being based on adeno-associated viral vectors. Adeno-associated virus (AAV) is a replication-deficient parvovirus, the single-stranded DNA genome of which is about 4.7 kb in length. The AAV genome includes inverted terminal repeat (ITRs) at both ends of the molecule, flanking two open reading frames: rep and cap. The cap gene encodes three capsid proteins: VP1, VP2 and VP3. The three capsid proteins typically assemble in a ratio of 1:1:8-10 to form the AAV capsid, although AAV capsids containing only VP3, or VP1 and VP3, or VP2 and VP3, have been produced. The cap gene also encodes the assembly activating protein (AAP) from an alternative open reading frame. AAP promotes capsid assembly, acting to target the capsid proteins to the nucleolus and promote capsid formation. The rep gene encodes four regulatory proteins: Rep78, Rep68, Rep52 and Rep40. These Rep proteins are involved in AAV genome replication.

The ITRs are involved in several functions, in particular integration of the AAV DNA into the host cell genome, as well as genome replication and packaging. When AAV infects a host cell, the viral genome can integrate into the host's chromosomal DNA resulting in latent infection of the cell. Thus, AAV can be exploited to introduce heterologous sequences into cells. In nature, a helper virus (for example, adenovirus or herpesvirus) provides protein factors that allow for replication of AAV in the infected cell and packaging of new virions. In the case of adenovirus, genes E1A, E1B, E2A, E4 and VA provide helper functions. Upon infection with a helper virus, the AAV provirus is rescued and amplified, and both AAV and the helper virus are produced.

AAV vectors (also referred to as recombinant AAV or rAAV) that contain a genome that lacks some, most or all of the native AAV genome and instead contains one or more heterologous sequences flanked by the ITRs have been successfully used in gene therapy and gene editing settings. These AAV vectors are widely used to deliver heterologous nucleic acid to cells of a subject. In some instances, the vectors are designed so that the heterologous nucleic acid integrates into the genome through homologous recombination (HR). For example, ex vivo engineering of cells, such as for immunotherapy, is of increasing interest and AAV vectors have been used for the purpose of integrating heterologous nucleic acid into a host cell for use in immunotherapy. For example, AAV vectors have been used to produce chimeric antigen receptor (CAR) T cells, where the nucleic acid encoding the CAR is delivered to the T cell ex vivo and HR-mediated gene editing results in the CAR nucleic acid being integrated at a specific gene locus (see e.g. Eyquem et al., 2017, Nature 543:113-119). Similarly, AAV vectors have been used to target, and integrate via ex vivo HR, heterologous sequences in hematopoietic stem and progenitor cells (HSPCs) (see e.g., Dever et al., 2016, Nature, 539:384-389). In both T-cells and HSPCs, the AAV-delivered sequences are integrated with the help of a double-strand break induced by targeted nucleases such as CRISPR/Cas9.

Although several AAV vectors have been utilised for HR-mediated gene editing, they are not necessarily efficient or effective. There is therefore a need to develop alternative methods for selecting and identifying AAV vectors that facilitate efficient integration of heterologous sequences into a genome via HR.

### SUMMARY OF THE DISCLOSURE

The present disclosure describes methods useful for AAV-directed evolution where selection is based on levels of HR-mediated editing of a distinct genetic locus. The selection platforms described herein therefore enable selection and identification of AAV vectors that facilitate robust HR-mediated gene editing in a host cell or tissue of choice. The disclosure also provides the necessary components for carrying out the methods, including replication-incompetent AAV and AAV libraries for use in the methods, and nucleic acid molecules to generate the replication-incompetent AAV particles and AAV libraries.

Thus, in one aspect, provided is a method for identifying an AAV cap gene from an AAV cap library, comprising the following steps:
a) transducing host cells with a test library of replication-incompetent AAV, wherein the replication-incompetent AAV comprise an AAV genome comprising two AAV ITRs flanking a left homology arm and a right homology arm, between which is an AAV cap gene from the AAV cap library, and wherein the sequence of the left homology arm and the sequence of the right homology arm are homologous to sequences at a locus in the genomic DNA of a host cell;
b) isolating genomic DNA from the one or more host cells from step a);
c) detecting integration of the cap gene into the host cell genomic DNA at a position in the locus; and
d) recovering the cap gene from the locus in the genomic DNA, thereby identifying an AAV cap gene, wherein the cap gene is suitable for the production of an AAV vector that can facilitate homologous recombination-mediated gene editing.

In some examples, the cap gene is operably linked to a promoter, such as a ubiquitous and/or constitutive promoter (e.g. a promoter selected from spleen focus forming virus (SFFV) promoter, Rous sarcoma virus (RSV) LTR promoter, the cytomegalovirus (CMV) promoter, the SV40 promoter, the dihydrofolate reductase promoter, the β-actin promoter, the phosphoglycerol kinase (PGK) promoter, the elongation factor-1 alpha promoter (EF-1α), and the short elongation factor-1 alpha promoter (EFS). In a particular example, the promoter is an AAV promoter (e.g. p40).

In particular embodiments, the AAV genome comprises an intron between the promoter and the cap gene. In one embodiment, the AAV genome comprises a poly adenylation sequence downstream of the cap gene.

In some examples, step c) comprises amplifying a sequence that comprises i) all or a portion of the cap gene and ii) at least a portion of the locus, wherein the portion of the gene is upstream of the left homology arm or downstream of the right homology arm. In particular embodiments, the amplifying utilises: i) a forward primer that is complementary to a region of the locus upstream of the left homology arm and a reverse primer that is complementary to the cap gene; ii) a forward primer that is complementary to the cap gene and a reverse primer that is complementary to a region of the locus downstream of the right homology arm; or iii) a forward primer that is complementary to a region of the locus upstream of the left homology arm and a reverse primer that is complementary to a region of the locus downstream of the right homology arm.

In some examples, the cap gene comprises a 3′ UTR and/or a 5′ UTR.

In one embodiment, the method further comprises a step of exposing the host cells to a genome editing nuclease before step a) or between step a) and step b). The genome editing nuclease may be, for example, selected from a zinc-finger nuclease (ZFN), transcription activator-like effector nucleases (TALEN) and clustered regularly interspaced short palindromic repeat (CRISPR)-Cas-associated nuclease. In one example, the step of exposing the host cells to a genome editing nuclease comprises exposing the host cells to a ribonucleoprotein complex comprising a CRISPR-Cas-associated nuclease (e.g. a Cas3, Cas9, Cas12 (e.g., Cas12a, Cas12b, Cas12c, Cas12d, Cas12e) or Cas14) and a guide RNA (gRNA).

In one example, the method further comprises a preselection process prior to step a), wherein the preselection process comprises selecting cap genes from the cap library on the basis of their ability to facilitate functional transduction of the host cells, wherein the selected cap genes are used to produce the test library of step a). In one embodiment, the preselection process comprises the following steps: i) transducing host cells with a library of replication-incompetent AAV, wherein the replication-incompetent AAV comprise a genome comprising two AAV ITRs flanking a reporter gene and a cap gene from the cap library; ii) selecting a plurality of host cells in which expression of the reporter gene is detected; iii) isolating RNA and optionally DNA from the plurality of host cells from step ii); iv) detecting reporter gene or cap gene mRNA; v) recovering the cap genes from the RNA or the DNA, or from cDNA produced from the mRNA, thereby identifying a plurality of cap genes that can facilitate functional transduction of the host cells; and vi) producing a plurality of replication-incompetent AAV with the plurality of cap genes so as to produce the test library of step a). In some examples, the reporter gene is operably linked to a first promoter (such as a ubiquitous and/or constitutive promoter, e.g. a promoter selected from spleen focus forming virus (SFFV) promoter, Rous sarcoma virus (RSV) LTR promoter, the cytomegalovirus (CMV) promoter, the SV40 promoter, the dihydrofolate reductase promoter, the B-actin promoter, the phosphoglycerol kinase (PGK) promoter, the elongation factor-1 alpha promoter (EF-1α), and the short elongation factor-1 alpha promoter (EFS)) and the cap gene is operably linked to a second promoter. In one example, the second promoter is an AAV promoter.

In some embodiments, recovering the cap gene in step d) comprises amplification of the cap gene. In particular examples, a plurality of cap genes are recovered in step d), optionally further comprising e) producing a plurality of replication-incompetent AAV as defined in step a) with the plurality of cap genes. In particular embodiments, the methods further comprise producing one or more AAV vectors using the one or more cap genes identified. Thus, also provided is an AAV vector produced by such methods.

In some examples, the host cell is an immune cell (e.g. T cells (including cytotoxic T cells, helper T cells, regulatory T cells, γδ T cells, αβ T cells, and mucosal-associated invariant T (MAIT) cells), natural killer (NK) cells, B cells, and dendritic cells (DC)), stem cell (e.g. hematopoietic stem and progenitor cells (HSPC), induced pluripotent stem cells (iPSC), neural stem and progenitor cells (NSPC), basal (lung) stem cells or mesenchymal stem cells (MSC)) or hepatocyte. In some embodiments, the locus is the TRAC, TRBC1, HLA, CD52, PD-1, IL2Ra, B2M, CD7, CTLA-4, WAS, IL7R, RAG1 a/b, CCR5, BTK, or ALB locus.

Also provided is a nucleic acid molecule, comprising an AAV genome comprising two AAV ITRs flanking a left homology arm and a right homology arm, between which is an AAV cap gene. In some examples, the cap gene comprises a 3′ UTR. Also provided is a combination comprising the afore-mentioned nucleic acid molecule and a further nucleic acid molecule comprising a rep gene operably linked to a promoter. In one embodiment, the combination further comprises a nucleic acid molecule comprising Adenovirus helper functions, or an Adenovirus.

Also provided is a plurality of nucleic acid molecules, wherein each nucleic acid molecule in the plurality comprises an AAV genome comprising two AAV ITRs flanking a left homology arm and a right homology arm, between which is an AAV cap gene. In some examples, at least 80%, 81%, 82%, 83%, 84%, 85%, 86%, 87%, 88%, 89%, 90%, 91%, 92%, 93%, 94%, 95%, 96%, 97%, 98% or 99% of the nucleic acid molecules in the plurality comprise a cap gene with a unique nucleic acid sequence relative to other cap genes in the plurality.

### BRIEF DESCRIPTION OF THE DRAWINGS

Embodiments of the disclosure are described herein, by way of non-limiting example only, with reference to the following drawings.

FIG. 1 is a schematic representation of a selection platform based on homologous recombination. A). A schematic of the genome of replication-incompetent AAV used in the Homologous Recombination (HR) selection platform, using homology arms (HA) targeting the T cell receptor alpha constant (TRAC) region of the human genome. The homology arms flank a capsid library expression cassette including the 154 nt AAV2 p40 promoter [wtAAV2 genome, NC_001401, 1700-1853nt] and the AAV2 intron followed by SwaI and NsiI restriction site-flanked capsid libraries of choice. B). A schematic of the genome of replication-incompetent AAV used in the HR selection platform using homology arms (HA) targeting the Bruton tyrosine kinase (BTK) region of the human genome. The homology arms flank a capsid library expression cassette including the 154 nt AAV2 p40 promoter and the AAV2 intron followed by SwaI and NsiI restriction site-flanked capsid libraries of choice. C). General layout of the capsid selection using the HR selection platform in any iteration. Cells are transduced with the library, the capsid gene is inserted into the target locus via HR and can be recovered by PCR, and then used to generate an AAV vector that supports HR-mediated gene editing.

FIG. 2 is a schematic representation of the two-round selection process incorporating a preselection step based on the FT platform followed by an HR-based selection. A) The FT platform was used to preselect capsids that facilitated functional transduction of T cells. A library of replication-incompetent AAV is transduced into cells. The replication-incompetent AAV contain a genome comprising ITRs flanking a cap gene from a capsid library (exemplified here as AAV2 or AAV6 peptide display libraries) operably linked to the p40 promoter and a reporter gene (eGFP) operably linked to the SFFV promoter. Following transduction and three-day culture of the T cells, RNA is extracted. Optionally, GFP-positive cells can be sorted using FACS. The RNA is converted to cDNA, and the cap genes (or the peptide portions in the exemplified embodiment here) are detected and amplified from the cDNA directly. Cap genes are recovered and then used to generate more replication-incompetent AAV for the next round. B) The second round (2a) was performed just like round 1 one using the secondary libraries in the FT platform. C) The second round (2b) of selection utilises the TRAC HR platform. T cells are electroporated using a spCas9/gRNA ribonucleoprotein (RNP) complex, before being transduced with a library of replication-incompetent AAV. The replication-incompetent AAV contain a genome comprising ITRs flanking a left TRAC HA and a right TRAC HA, which themselves flank a cap gene from a capsid library operably linked to the p40 promoter. Genomic DNA is isolated from the T cells and integration of the cap gene by HR is detected by PCR. D) The second round (2c) of selection utilises the BTK HR platform. Hematopoietic stem cells (HSCs) are electroporated using a spCas9/gRNA ribonucleoprotein (RNP) complex, before being transduced with a library of replication-incompetent AAV. The replication-incompetent AAV contain a genome comprising ITRs flanking a left BTK HA and a right BTK HA, which themselves flank a cap gene from a capsid library operably linked to the p40 promoter. Genomic DNA is isolated from the HSCs and integration of the cap gene by HR is detected by PCR. E) Integration of selection 2c is quantified by left homology arm spanning ddPCR and confirming functionality of all libraries in three different HSC donors.

FIG. 3 is a schematic representation of the PCR recovery method for integrated peptide display capsid genes and strategy to validate specificity of the PCR. A). PCR strategy to recover integrated capsid genes coding for an AAV peptide display variant. The forward (fwd) primer binds outside the left HA; the reverse (rev) primer binds shortly behind the peptide coding region. When using the TRAC HR platform for selection in T cells together with the exemplified peptide display library, the expected amplicon length is 2.8 kb. This fragment can be further digested with blunt cutting restriction enzymes such as MscI to create a short fragment which can be submitted for PE150 Illumina sequencing. B). Scenarios and PCR templates from differently transduced T cells to validate the specificity of the PCR strategy to only amplify correctly HR-integrated capsid genes. (I) DNA from SpCas9 RNP-electroporated T cells transduced with a pre-selected TRAC-HR.AAV6 peptide display library serving as the validation template as this is the sample most likely to get correctly integrated. (II) DNA from SpCas9 RNP-electroporated T cells transduced with a pre-selected FT.AAV6 peptide display library serving as a negative control as targeted integration of the library is not expected. (III) DNA from untreated T cells transduced with a pre-selected TRAC-HR.AAV6 peptide display library serving as a control to test if the SpCas9-mediated DNA break is necessary for selection. (IV) Untreated control T cells serving as an additional negative control. C) The gel image shows amplicons from each of conditions I-IV, using DNA extracted from the T cells using a phenol chloroform precipitation method or a NEB HMW DNA extraction kit.

FIG. 4 demonstrates a PCR recovery method for integrated peptide display capsid genes and strategy to validate specificity of the PCR. A) PCR strategy to recover integrated full length capsid genes coding for an AAV shuffled library or other complete mutations such as domain swaps and random mutagenesis. The forward (fwd) primer binds outside the transgene encoded homology arm (HA); the reverse (rev) primer binds shortly behind the VP1 CDS. When using the TRAC HR platform for selection in T cells together with any AAV capsid library, the expected amplicon length is 3.2 kb. This fragment can be further digested with blunt cutting restriction enzymes such as SwaI to create a fragment which can be subcloned for vectorization and sequence identification. B) Bulk PCR showing specific amplification of the target amplicon from DNA of SpCas9 RNP-electroporated T cells transduced with a pre-selected TRAC-HR.AAV4/AAV6 shuffled library.

FIG. 5 is a graphical representation showing position-specific amino acid enrichment for AAV2 and AAV6 peptide display libraries using the TRAC-HR selection platform. A) AAV2 peptide display library amino acid enrichment per position. B) AAV6 peptide display library amino acid enrichment per position. The bars are color coded based on the chemical properties of the amino acid residues. A theoretical consensus sequence for both selections is shown below.

FIG. 6 demonstrates individual testing of novel AAV variants identified from three capsid libraries using the HR platform. A) A schematic representation of the study. T cells were electroporated with the Cas9/gRNA RNP complexes, followed by individual AAV transduction at a dose of 10,000 vg/cell. The AAVs packaged promoter-less GFP flanked by TRAC HAs, enabling GFP expression following successful integration into the TRAC locus. Levels of HR (or homology directed repair; HDR) were evaluated 14 days after treatment by flow cytometry. Additionally, T-cells were counted to determine potential toxicity from each AAV. B) Homologous recombination efficiency versus T cell expansion of novel AAV variants. AAV6 was used as the benchmark and all GFP expression (Y-axis) was adjusted to it. The strongest expansion of T-cells was observed after transduction of ‘No AAV’ and AAV2, AAV4 & AAV12, hence the ‘No AAV’ control was used to adjust the expansion values (X-axis). The area drawn includes all variants that are mediating 50% higher expansion and 25% higher HDR than AAV6.

FIG. 7 provides data to support the near normal distribution of all 200 AAV2-based peptide insertion variants, 200 AAV6-based peptide insertion variants, and spike-in AAV6 control. A) The data was derived from vectorized viral genomes. B-C) show the distribution of variants in the FT-eGFP selection platform. D-E) show distributions of TRAC HR encoded AAV2 and AAV6 peptide libraries. F-G) show distributions of BTK HR encoded AAV2 and AAV6 peptide libraries.

FIG. 8 demonstrates the functionality of synthetic oligonucleotide-generated peptide display libraries and selection stringencies of available recovery methods. A) GFP expression from successful FT library transduction into primary human T cells. B) PCR rescued integrated capsid genes from successfully gene edited primary human T cells using the TRAC embodiment of the HR platform. C-D) All data was generated from independently performed experiments in cells from four donors. (C) Capsid performance enrichment scores

$$( \frac{NGS contribution after selection}{NGS contribution after manufacturing} )$$

is plotted on the y-axis for FT platform readouts total DNA, nuclear DNA, and RNA expression; and for AAV-mediated HDR performance using the TRAC-HR platform. (D) Table showing lowest, highest, average/mean, and median values for each selection enrichment scores. Selection stringency was determined as skewing

$$( skew = \frac{mean}{median} ) .$$

FIG. 9 shows a comparison of selection pressures using the synthetic libraries in the FT- and HR selection platforms in T-cells. All data was generated from independently performed experiments in cells from four donors. Analysis was based on the same data as FIG. 8 C-D but organised by rank of capsid performance facilitating a better comparison between platforms. (a) Synthetic oligonucleotide-coded peptide insertion capsid performance rank based on total DNA entry, nuclear DNA, and RNA expression on the y-axis. Y-axis data was generated using recovery from the synthetic library in the FT platform. HDR performance recovered from selections using the TRAC-HR platform plotted on the x-axis. (b) Details of the ordinal linear regression analysis, such as the equation, whether the slope is significantly not zero, correlation between the FT read-out and HDR performance (R and R²).

FIG. 10 shows a comparison of selection pressures using the most highly T-cell-enriched capsids from the synthetic libraries based on DNA, nuclear DNA, and RNA. Analysis was based on the same data as in FIG. 9 but filtered for the 25 most enriched capsids for each of the three recovery methods for the FT platform, resulting in 54 capsids in total. (a) Synthetic oligonucleotide-coded peptide insertion capsid performance rank based on total DNA entry, nuclear DNA, and RNA expression on the y-axis. Y-axis data was generated using recovery from the synthetic library in the FT platform. HDR performance recovered from selections using the TRAC-HR platform plotted on the x-axis. (b) Details of the ordinal linear regression analysis, such as the equation, whether the slope is significantly not zero, correlation between the FT read-out and HDR performance (R and R²).

FIG. 11 shows the enrichment of novel capsids over AAV6 in synthetic libraries selected for RNA expression and HDR efficiency in T-cells. Analysis was based on the same data as in FIG. 8 C-D but normalised to the capsid performance enrichment score of spiked-in AAV6. (a) Synthetic oligonucleotide-coded peptide insertion capsid performance based on RNA expression using the synthetic library in the FT platform on the y-axis. Data for AAV2- and AAV6-derived variants shown separately. (b) Synthetic oligonucleotide-coded peptide insertion capsid performance based on AAV-mediated HDR performance using the synthetic library in the TRAC-HR platform on the y-axis. Data for AAV2-and AAV6-derived variants shown separately. All experiments were performed in four independent donors. Statistics: non-parametric Mann-Whitney test: ****<0.0001

FIG. 12 demonstrates multiplexed high-throughput parallel testing of novel AAV variants. A) A schematic representation of the study. AAV6 and 24 novel AAVs were produced to package a barcoded promoterless GFP flanked by TRAC homology arms. An equimolar mix of these 25 variants was created which is compatible with high-throughput parallelised analysis of AAV entry (PCR from total DNA of T-cells that were transduced with the AAV mix, but not electroporated with RNPs) and AAV-mediated HDR by performing a PCR with primer binding sites inside the transgene and outside the homology arms. In addition to NGS analysis, cells were flow analysed for GFP expression to evaluate the overall functionality of the AAV mix, as well as allele targeting by the RNP shown as a reduction of TCR alpha-detection by APC-labelled antibodies. B) Flow cytometry analysis showing the functionality of the AAV mix and TCR disruption efficiency of RNPs.

FIG. 13 is a graphical representation of the performance of the novel AAV variants when transduced as a competitive NGS mix. AAV mediated HDR (Y-axis) was adjusted to the performance of AAV6. The X-axis shows the entry efficiency of the novel AAVs in primary human T cells A) Low dose of the AAV NGS mix. B) High dose of the AAV NGS mix. The top candidates are encircled, and all contain AAV6-P05 (top performer in FIG. 11b) alongside other AAV6 modifications.

FIG. 14 shows the selection stringency of various recovery methods in HSPCs. All data was generated from independently performed experiments in cells from four donors. (A) Capsid performance enrichment scores

$$( \frac{NGS contribution after selection}{NGS contribution after manufacturing} )$$

is plotted on the y-axis for FT platform readouts total DNA, nuclear DNA, and RNA expression; and for AAV-mediated HDR performance using the TRAC-HR platform. (B) Table showing lowest, highest, average/mean, and median values for each selection enrichment scores. Selection stringency was determined as skewing

$$( skew = \frac{mean}{median} ) .$$

FIG. 15 shows comparison of selection pressures using the synthetic libraries in the FT- and HR selection platforms in HSPCs. All data was generated from independently performed experiments in cells from four donors. Analysis was based on the same data as FIG. 14 but organised by rank of capsid performance facilitating a better comparison between platforms. (a) Synthetic oligonucleotide-coded peptide insertion capsid performance rank based on total DNA entry, nuclear DNA, and RNA expression on the y-axis. Y-axis data was generated using recovery from the synthetic library in the FT platform. HDR performance recovered from selections using the BTK-HR platform plotted on the x-axis. (b) Details of the ordinal linear regression analysis, such as the equation, whether the slope is significantly not zero, correlation between the FT read-out and HDR performance (R and R²).

FIG. 16 shows comparison of selection pressures using the most highly HSPC-enriched capsids from the synthetic libraries based on DNA, nuclear DNA, and RNA. Analysis was based on the same data as in FIG. 15 but filtered for the 25 most enriched capsids for each of the three recovery methods for the FT platform, resulting in 56 capsids in total. (a) Synthetic oligonucleotide-coded peptide insertion capsid performance rank based on total DNA entry, nuclear DNA, and RNA expression on the y-axis. Y-axis data was generated using recovery from the synthetic library in the FT platform. HDR performance recovered from selections using the BTK-HR platform plotted on the x-axis. (b) Details of the ordinal linear regression analysis, such as the equation, whether the slope is significantly not zero, and the correlation between the FT read-out and HDR performance (R and R²).

FIG. 17 shows the enrichment of novel capsids over AAV6 in synthetic libraries selected for RNA expression and HDR efficiency in HSPCs. Analysis was based on the same data as in FIG. 14 but normalised to the capsid performance enrichment score of spiked-in AAV6. (a) Synthetic oligonucleotide-coded peptide insertion capsid performance based on RNA expression using the synthetic library in the FT platform on the y-axis. Data for AAV2- and AAV6-derived variants shown separately. (b) Synthetic oligonucleotide-coded peptide insertion capsid performance based on AAV-mediated HDR performance using the synthetic library in the BTK-HR platform on the y-axis. Data for AAV2- and AAV6-derived variants shown separately. All experiments were performed in four independent donors. Statistics: non-parametric Mann-Whitney test: ****<0.0001

### DETAILED DESCRIPTION

Unless defined otherwise, all technical and scientific terms used herein have the same meaning as is commonly understood by one of skill in the art to which the disclosure belongs. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

As used herein, the singular forms “a”, “an” and “the” also include plural aspects (i.e. at least one or more than one) unless the context clearly dictates otherwise. Thus, for example, reference to “a polypeptide” includes a single polypeptide, as well as two or more polypeptides.

In the context of this specification, the term “about,” is understood to refer to a range of numbers that a person of skill in the art would consider equivalent to the recited value in the context of achieving the same function or result.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “host cell” refers to a cell, such as a mammalian cell, that has exogenous DNA introduced into it, such as a vector or other polynucleotide. The term includes the progeny of the original cell into which the exogenous DNA has been introduced. Thus, a “host cell” as used herein generally refers to a cell that has been transfected or transduced with exogenous DNA.

As used herein, a “vector” includes reference to both polynucleotide vectors and viral vectors, each of which are capable of delivering a transgene contained within the vector into a host cell. Vectors can be episomal, i.e., do not integrate into the genome of a host cell, or can integrate into the host cell genome. The vectors may also be replication competent or replication-deficient. Exemplary polynucleotide vectors include, but are not limited to, plasmids, cosmids and transposons. Exemplary viral vectors include, for example, AAV, lentiviral, retroviral, adenoviral, herpes viral and hepatitis viral vectors.

As used herein, “adeno-associated viral vector” or AAV vector refers to a vector in which the capsid is derived from an adeno-associated virus, including without limitation, AAV1, AAV2, AAV3, AAV4, AAV5, AAV6, AAV7, AAV8, AAV9, AAV10, AAV11, AAV12 or AAV13, or using synthetic or modified AAV capsid proteins, including chimeric capsid proteins. When referring to AAV vectors, both the source of the genome and the source of the capsid can be identified, where the source of the genome is the first number designated and the source of the capsid is the second number designated. Thus, for example, a vector in which both the capsid and genome are derived from AAV2 is more accurately referred to as AAV2/2. A vector with an AAV6-derived capsid and an AAV2-derived genome is most accurately referred to as AAV2/6. A vector with the synthetic DJ capsid and an AAV2-derived genome is most accurately referred to as AAV2/DJ. For simplicity, and because most vectors use an AAV2-derived genome, it is understood that reference to an AAV6 vector generally refers to an AAV2/6 vector, reference to an AAV2 vector generally refers to an AAV2/2 vector, etc. An AAV vector may also be referred to herein as “recombinant AAV”, “rAAV”, “recombinant AAV virion”, and “rAAV virion,” terms which are used interchangeably and refer to a replication-defective virus that includes an AAV capsid shell encapsidating an AAV genome. The AAV vector genome (also referred to as vector genome, recombinant AAV genome or rAAV genome) comprises a transgene flanked on both sides by functional AAV ITRs. Typically, one or more of the wild-type AAV genes have been deleted from the genome in whole or part, preferably the rep and/or cap genes. Functional ITR sequences are necessary for the rescue, replication and packaging of the vector genome into the rAAV virion.

The term “ITR” refers to an inverted terminal repeat at either end of the AAV genome. This sequence can form hairpin structures and is involved in AAV DNA replication and rescue, or excision, from prokaryotic plasmids. ITRs for use in the present disclosure need not be the wild-type nucleotide sequences, and may be altered, e.g., by the insertion, deletion or substitution of nucleotides, as long as the sequences provide for functional rescue, replication and packaging of rAAV.

As used herein, the term “operably-linked” with reference to a promoter and a coding sequence means that the transcription of the coding sequence is under the control of, or driven by, the promoter.

The term “reporter gene” as used herein refers to a gene which encodes a gene product suitable for screening or sorting cells transduced with an AAV described herein that contains a genome comprising the reporter gene. The gene product can be any polypeptide or protein suitable for the intended use for screening technologies and can be cytoplasmic or membrane-bound. To facilitate sorting, the gene product can be directly detectable (e.g. may be a fluorescent protein), or may be indirectly-detectable, such as by using a labelled antibody that binds to the gene product. For the purposes of the present disclosure, the reporter gene does not encode an AAV capsid.

By “complementary” it is meant that a nucleic acid (e.g., RNA, DNA) comprises a sequence of nucleotides that enables it to non-covalently bind, i.e., form Watson-Crick base pairs and/or G/U base pairs, “anneal”, or “hybridize” to another nucleic acid in a sequence-specific, antiparallel, manner (i.e., a nucleic acid specifically binds to a complementary nucleic acid) under the appropriate in vitro and/or in vivo conditions of temperature and solution ionic strength. Reference to “complementary” does not require complete or 100% complementarity, but can include less than complete or less than 100%, such as 70%, 75%, 80%, 85%, 90% or 95% complementarity. Standard Watson-Crick base-pairing includes: adenine/adenosine (A) pairing with thymidine/thymidine (T), A pairing with uracil/uridine (U), and guanine/guanosine (G) pairing with cytosine/cytidine (C). In addition, for hybridization between two RNA molecules (e.g., dsRNA), and for hybridization of a DNA molecule with an RNA molecule (e.g., when a target nucleic acid sequence base pairs with a gRNA) G can also base pair with U. For example, G/U base-pairing is partially responsible for the degeneracy (i.e., redundancy) of the genetic code in the context of tRNA anti-codon base pairing with codons in rnRNA. Thus, in the context of this disclosure, a G (e.g., of a target nucleic acid sequence base pairing with a gRNA) is considered complementary to both a U and to C. For example, when a G/U base-pair can be made at a given nucleotide position of a protein binding segment of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tₘ) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

By “gene” it is meant a unit of inheritance that, when present in its endogenous state, occupies a specific locus on a genome and comprises transcriptional and/or translational regulatory sequences and/or a coding region and/or non-translated sequences (i.e., introns, 5′ and 3′ untranslated sequences).

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The terms “protein”, “peptide” and “polypeptide” are used interchangeably herein to refer to a polymer of amino acid residues linked together by peptide (amide) bonds. The terms refer to a protein, peptide, or polypeptide of any size, structure or function.

As used herein, genome editing refers to the modification of the sequence of a host cell genome. The modification can include insertion and/or deletion of one or more nucleotides, and/or substitution or replacement of one or more nucleotides. Genome editing can be performed in vitro, in vivo or ex vivo.

The term “genome editing nuclease” refers to any enzyme that can catalyze the cleavage of phosphodiester bonds in nucleic acid, thereby facilitating or supporting genome editing.

The terms “guide RNA” and “gRNA” refer to a RNA sequence that is complementary to a target nucleic acid sequence and directs a RNA-guided nuclease to the target nucleic acid sequence. gRNA typically comprises CRISPR RNA (crRNA) and a tracr RNA (tracrRNA). “crRNA” is a 17-20 nucleotide sequence that is complementary to the target nucleic acid sequence, while the “tracrRNA” provides a binding scaffold for the RNA-guided nuclease. crRNA and tracrRNA exist in nature as two separate RNA molecules, which have been adapted for molecular biology techniques using, for example, 2-piece gRNAs such as CRISPR tracer RNAs (cr:tracrRNAs).

The terms “single-guide RNA” and “sgRNA” refer to a single RNA sequence that comprises the crRNA fused to the tracrRNA. Accordingly, the skilled person would understand that the term “gRNA” describes all CRISPR guide formats, including two separate RNA molecules or a single RNA molecule. By contrast, the term “sgRNA” will be understood to refer to single RNA molecules combining the crRNA and tracrRNA elements into a single nucleotide sequence.

As used herein, a “homology arm” refers to a nucleic acid region or segment that has a sequence that is homologous to a genome on one or both sides of a target site in a genome locus, such that homologous recombination can occur between the genome and the homology arm, resulting in insertion of nucleic acid present between two homology arms at the target site, and/or removal of the equivalent nucleic acid from the native genome. The homology arms may have complete homology (i.e. 100% homology or sequence identity) or may have partial homology (e.g. 90%, 91%, 92%, 93%, 94%, 95%, 96%, 97%, 98% or 99% homology or sequence identity) to a sequence in the genome.

The phrase “supports HR-mediated gene editing” or grammatical variants thereof with respect to an AAV vector, AAV capsid polypeptide or AAV cap gene means that the AAV vector, or an AAV vector produced with the AAV capsid polypeptide or cap gene (i.e. an AAV vector comprising a capsid comprising the capsid polypeptide or a capsid polypeptide encoded by the cap gene), can be used to deliver nucleic acid that can be incorporated into a host cell genome through a homologous recombination event. Integration of the nucleic acid into the genome through homologous recombination can be in the presence or absence of a genome editing nuclease. In some instances, the level or frequency of the HR-mediated gene editing that is supported by the AAV vector, AAV capsid polypeptide or cap gene is increased compared to a reference AAV vector or AAV capsid, such as by 20%, 30%, 40%, 50%, 60%, 70%, 80%, 90%, 100%, 150%, 200%, 250%, 300% or more.

It will be appreciated that the above described terms and associated definitions are used for the purpose of explanation only and are not intended to be limiting.

TABLE 1
Brief Description of the Sequences

| SEQ ID NO. | Description |
|---|---|
| 1 | VG1228_p40-AAVintron_F |
| 2 | VG1229_p40-AAVintron_R |
| 3 | VG1234_left-HA-TRAC-F |
| 4 | VG1235_left-HA-TRAC-R |
| 5 | VG1236_NsiIRHA-TRAC_F |
| 6 | VG1237_FseIRHA-TRAC-R |
| 7 | AAV ITR left |
| 8 | AAV ITR right |
| 9 | eGFP |
| 10 | SV40 polyA sequence |
| 11 | SFFV promoter |
| 12 | P40 promoter |
| 13 | SV40 intron |
| 14 | TRAC gRNA |
| 15 | AAV2/AAV6 peptide fwd primer |
| 16 | AAV2/AAV6 peptide rev primer |
| 17 | AAV4/AAV6 shuffled fwd primer |
| 18 | AAV4/AAV6 shuffled rev primer 1 |
| 19 | AAV4/AAV6 shuffled rev primer 2 |
| 20 | TRAC genomic fwd primer |
| 21 | BTK gRNA |
| 22 | BTK genomic forward primer |
| 23 | B-LHA_F |
| 24 | B-LHA_R |
| 25 | B-RHA_F |
| 26 | B-RHA_R |

### Homologous Recombination Selection Platform

The present disclosure is directed, in part, to methods for AAV directed evolution where the selection pressure is based on homologous recombination (HR)-mediated gene editing. The HR selection platform described herein can therefore be used to select and identify variant capsid genes, capsid polypeptides and AAV vectors that support HR-mediated gene editing, i.e. the variant capsid polypeptides encoded by the variant capsid genes can be used to produce variant AAV vectors that support HR-mediated gene editing. In some instances, the level or frequency of the HR-mediated gene editing that is supported by the variant AAV vector, AAV capsid polypeptide or cap gene is increased compared to a reference AAV vector, AAV capsid polypeptide or AAV cap gene (e.g. an AAV6 vector, capsid polypeptide or cap gene), such as by 20%, 30%, 40%, 50%, 60%, 70%, 80%, 90%, 100%, 150%, 200%, 250%, 300% or more.

The HR platform of the present disclosure utilises AAV containing two ITRs flanking a left homology arm and a right homology arm, between which is an AAV cap gene from an AAV cap library. The sequence of the left homology arm and the sequence of the right homology arm are homologous to sequences at a locus in the genomic DNA of a host cell, such as at a gene locus, such that the cap gene can be inserted or integrated into the genomic DNA at the locus via homologous recombination between the homology arms and the genomic DNA. Integration of the cap gene can then be detected by, for example, PCR amplification using a primer specific for the cap gene and a primer specific for the genomic DNA upstream or downstream of the locus at which the cap gene was integrated.

Thus, in one aspect, provided is a method for identifying an AAV cap gene from an AAV cap library, comprising: a) transducing host cells with a test library of replication-incompetent AAV, wherein the replication-incompetent AAV comprise an AAV genome comprising two AAV ITRs flanking a left homology arm and a right homology arm, between which is an AAV cap gene from the AAV cap library, and wherein the sequence of the left homology arm and the sequence of the right homology arm are each homologous to sequences at a locus (e.g. a gene) in the genomic DNA of one or more host cells; b) isolating genomic DNA from the one or more host cells from step a); c) detecting integration of the cap gene into the host cell genomic DNA at the locus (e.g. at a position in the gene); and d) recovering the cap gene from the locus in the genomic DNA, thereby identifying an AAV cap gene. In particular embodiments, a further step of exposing the host cells to a genome editing nuclease is included before step a), or between step a) and step b).

As would be appreciated, the AAV cap gene identified as a result of the HR selection platform is suitable for the production of an AAV vector that can facilitate or support HR-mediated gene editing of a cell, such as a cell of the same general type as the host cell used for selection. Exemplary host cells include any type of replicating cell. In one example, the cells are immune cells, such T cells (including αβ T cells, cytotoxic T cells, helper T cells, regulatory T cells, γδ T cells, and mucosal-associated invariant T (MAIT) cells), natural killer (NK) cells, B cells, and dendritic cells (DC). In other examples, the host cells are stem cells, such as hematopoietic stem and progenitor cells (HSPC), induced pluripotent stem cells (iPSC), neural stem and progenitor cells (NSPC), basal (lung) stem cells or mesenchymal stem cells (MSC). In further examples, the cells are hepatocytes.

Each of the homology arms (i.e. the left or 3′ homology arm, and the right or 5′ homology arm) have a sequence that is homologous to a sequence in the genomic DNA at the locus being targeted for HR-mediated gene editing. The left or 3′ homology arm is homologous to a region that is upstream of (or 3′ to) the targeted site of integration, while the right or 5′ homology arm is homologous to a region that is downstream of (or 5′ to) the targeted site of integration. The homology arms are therefore used to target the locus and facilitate or enable HR. For the purposes of the present disclosure, the homology arms are typically about 50 bp to about 800 bp, about 150 bp to about 750 bp, or about 300 bp to about 700 bp in length (e.g. so as to keep within the permitted or optimal genome length of the AAV genome).

The homology arms can be designed to be homologous to, and thus target the cap gene to, any desired locus in the genomic DNA of the host cell. In one example, the locus is the T-cell receptor α constant (TRAC) locus and the homology arms are homologous to regions in the TRAC gene. As described in Eyquem et al. (Nature, 2017, 543:113-119), targeting a chimeric antigen receptor (CAR) to this locus results in uniform expression of the CAR in human peripheral blood T cells and enhances T cell potency. Thus, selecting and identifying AAV variants that effectively support HR-mediated integration of a nucleic acid at the TRAC locus of a T cell would be of benefit for the ex vivo production of CAR T cells. Other loci that are particularly relevant to T cells include the T cell receptor β-chain constant region 1 (TRBC1), human leukocyte antigen (HLA), CD52, PD-1, IL2Ra, B2M, CD7, and CTLA-4 (for review, see Atsavapranee et al., 2021, EBioMedicine, 67:103354). In another example, the locus is one that is targeted with hematopoietic stem cells (HSC) gene therapy, whereby selecting and identifying AAV variants that effectively support HR-mediated integration of a nucleic acid at these loci would be of benefit for the ex vivo production of engineered HSC for gene therapy. Such loci include, for example, Bruton's tyrosine kinase (BTK) gene, interleukin-7 receptor (IL7R), RAG1A/B, CCR5, and the Wiskott-Aldrich syndrome (WAS). In a further example, the loci is the albumin (ALB) gene, which may be targeted when HR-mediated integration into the genomic DNA of a hepatocyte is desired.

Typically, the cap gene is operatively linked to a promoter that is functional in the host cell. In some examples, the promoter is constitutive in the host cells used for selection and potentially also for the downstream therapeutic applications. In particular examples, the promoter is a ubiquitous promoter (i.e. functional in multiple tissue types or multiple host cells) and/or a constitutive promoter. In other examples, the promoter is tissue-specific. Suitable promoters are well known to those skilled in the art and non-limiting examples are provided below.

In some embodiments of the HR selection platform, the selection process is performed in the absence of a genome editing nuclease. Typically however, a genome editing nuclease is utilised in the selection process such as before step a), or between step a) and step b) of the process as exemplified in [0060]. Exposure of the host cells to a genome editing nuclease, either before or after the cells are transduced with the test library of replication-incompetent AAV, enhances HR by inducing double-stranded breaks (DSBs) in the genomic DNA at the locus. These DSBs are then repaired by homology directed repair (HDR) using the templates of the homology arms, resulting in integration of the cap gene at the locus.

Any suitable genome editing nuclease can be used, including CRISPR-associated protein (Cas) endonucleases, zinc finger nucleases (ZFNs), transcription activator-like effector nucleases (TALENs), meganucleases, variants, fragments and combinations thereof. Naturally-occurring and synthetic genome editing nuclease are contemplated herein.

In one embodiment, the genome editing system is a CRISPR-Cas genome editing system. The “clustered regularly interspaced short palindromic repeat” (CRISPR)/“CRISPR-associated protein” (Cas) system (CRISPR/Cas system) evolved in bacteria and archaea as an adaptive immune system to defend against viral attack. The mechanisms of CRISPR-mediated gene editing would be known to persons skilled in the art and have been described, for example, by Doudna et al., (2014, Methods in Enzymology, 546). Briefly, upon exposure to a virus, short segments of viral DNA are integrated in the clustered regularly interspaced short palindromic repeats (i.e., CRISPR) locus. RNA is transcribed from a portion of the CRISPR locus that includes the viral sequence. That RNA, which contains sequence complementarity to the viral genome, mediates targeting of a Cas endonuclease to the sequence in the viral genome. The Cas endonuclease cleaves the viral target sequence to prevent integration or expression of the viral sequence. Suitable Cas endonucleases for the methods of the present disclosure would be known to persons skilled in the art, illustrative examples of which include Cas3, Cas9, Cas12 (e.g., Cas12a, Cas12b, Cas12c, Cas12d, Cas12e) and Cas14.

Thus, in one example, the host cells are exposed to a ribonucleoprotein (RNP) complex comprising a Cas endonuclease and suitable guide RNA (gRNA) specific for the loci. Methods and tools for the design of gRNA would be known to persons skilled in the art, illustrative examples of which include CHOPCHOP, CRISPR Design, sgRNA Designer, Synthego and GT-Scan. Suitable gRNAs would be known to persons skilled in the art or could be designed and produced by persons skilled in the art, illustrative examples of which include the gRNAs described elsewhere and herein, such as the gRNA targeting TRAC set forth in SEQ ID NO: 14 or the gRNA described in in Eyquem et al. (Nature, 2017, 543:113-119).

Exposure of the cells to the genome editing nuclease, including the RNP complex, can be by any suitable means, such as electroporation, nucleofection, and lipid-mediated transfection.

Following transduction of the host cells with the test library of replication-incompetent AAV and optionally exposure of the cells to a genome editing nuclease before or after said transduction, the host cells are cultured for a suitable time period (e.g., 1, 2, 3, 4, 5, 6, 7, 8, 9, 10 or more days) before the genomic DNA is isolated from the cells. Integration of the cap gene into the locus targeted by the homology arms is detected by, for example, PCR using a primer specific for the cap gene and a primer specific for a region in the locus that is adjacent to the integrated left or right homology arm (and is not present in the genome of the replication-incompetent AAV). The resulting amplicon will therefore comprise all or a portion of the cap gene and at least a portion of the locus that is not present in either the left or right homology arm. Thus, in some examples, the amplification utilises: i) a forward primer that is complementary to a region of the locus upstream of the left homology arm and a reverse primer that is complementary to the cap gene; ii) a forward primer that is complementary to the cap gene and a reverse primer that is complementary to a region of the locus downstream of the right homology arm; or iii) a forward primer that is complementary to a region of the locus upstream of the left homology arm and a reverse primer that is complementary to a region of the locus downstream of the right homology arm.

Cap genes that have been identified as being integrated into the locus are then recovered from the genomic DNA. Recovery of the cap gene can be performed by any manner known to those skilled in the art. Typically PCR amplification of the cap sequence is performed, such as by using primers specific for 5′ and 3′ regions of the cap gene, and/or primers specific for the upstream and downstream regions flanking the cap gene, e.g. the promoter (e.g. downstream of the transcriptional start site), 5′ UTR, 3′ UTR, or polyadenylation sequence, depending on the configuration of the vector genome. The recovered cap genes can optionally be used to generate a further library for a further round of selection, or may be vectorized so as to produce an AAV vector having a capsid encoded by the recovered cap gene. In some examples, other properties of the vectors, such as toxicity to the host cell and ability to support cell replication or expansion, are also assessed.

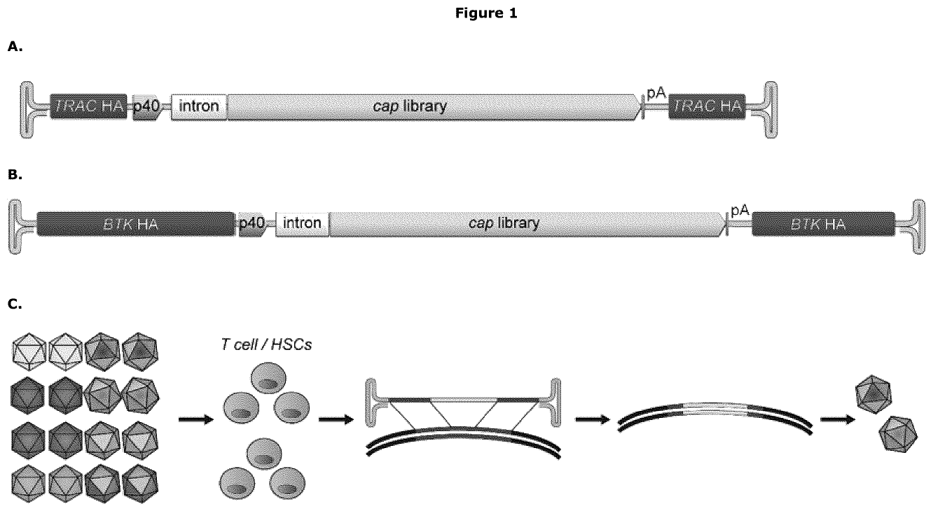

An exemplary HR selection process is shown in FIG. 1.

### Preselection using the Functional Transduction Platform

In some examples, a preselection process based on Functional Transduction (FT) is performed prior to the HR selection. This preselection process comprises selecting cap genes from the cap library on the basis of their ability to facilitate functional transduction of the host cells (i.e. transduction of, and transgene expression in, host cells). This ensures that any capsid identified in the HR selection process is also functional. The FT platform is described in detail in WO2020077411, and any variation of the FT platform can be used for the preselection process.

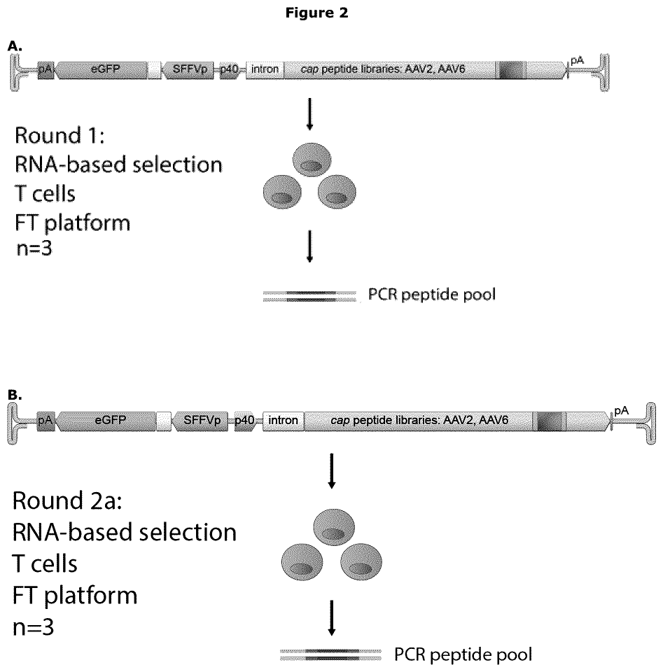

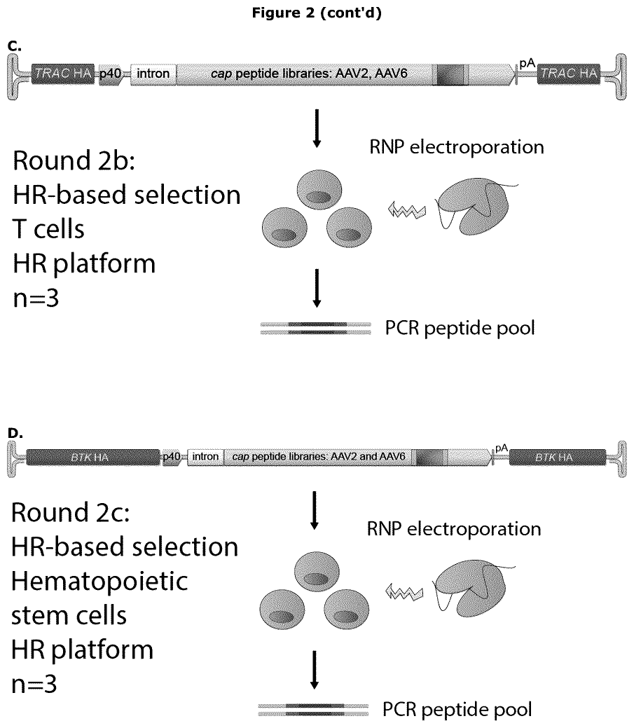

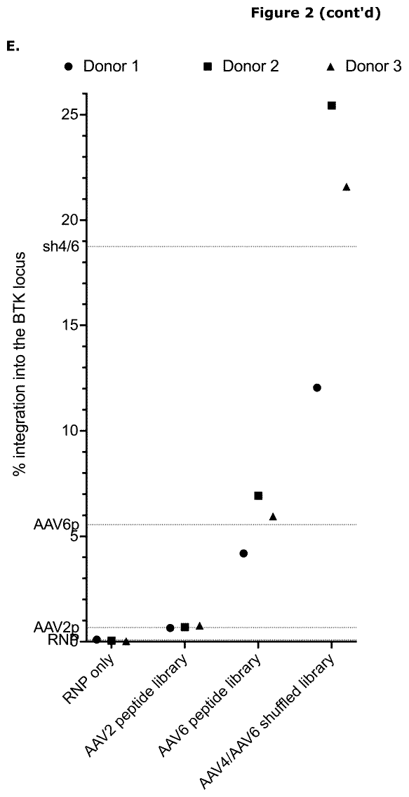

In one example, the preselection process comprises: i) transducing host cells with a library of replication-incompetent AAV, wherein the replication-incompetent AAV comprise a genome comprising two AAV ITRs flanking a reporter gene and a cap gene from the cap library; ii) selecting a plurality of host cells in which expression of the reporter gene is detected; iii) isolating RNA and optionally DNA from the plurality of host cells from step ii); iv) detecting reporter gene or cap gene mRNA; v) recovering the cap genes from the RNA or the DNA, or from cDNA produced from the mRNA, thereby identifying a plurality of cap genes that can facilitate functional transduction of the host cells; and vi) producing a plurality of replication-incompetent AAV with the plurality of cap genes so as to produce the test library then utilised in the HR selection process. This is shown schematically in FIG. 2.

The reporter gene can be any that encodes a polypeptide or protein that can be directly detected using a suitable sorting process, or that can be indirectly-detected using a suitable sorting process, such as by using a labelled antibody that binds to the gene product. Examples of fluorescent agents include blue, cyan, green, yellow, orange, red, and far-red fluorescent protein. Examples of luminescent agents include e.g. luciferase proteins (such as firefly, renilla, gaussia luciferase). Examples of cell surface markers include molecules from the cluster of differentiation (CD), artificial epitopes or membrane-bound proteins from agents mentioned above such as fluorescent proteins. The reporter gene product can be truncated or a fusion protein. In a one embodiment, the reporter gene is a fluorescent protein, such as green fluorescent protein (GFP), including eGFP.

Other common fluorescent proteins such as other green and red fluorescent proteins (ZsGreen1, tdTomato, DsRed, AsRed, mStrawberry, mCherry, mOrange), yellow fluorescent proteins (YFP, mBanana, ZsYellow1), cyan fluorescent proteins (CFP, AmCyan1) or blue fluorescent proteins (BFP) or far-red fluorescent proteins (mKate2, HcRed1, mRaspberry, E2-Crimson, mPlum) may also be used. In addition, the reporter gene may encode fluorescent proteins with enhanced brightness such as eXFP, TagXFP or TurboXFP, wherein X may be any green, red, yellow, blue or far-red fluorescent protein as mentioned above.

Means suitable for sorting cells, including for example, fluorescence-activated cell sorting (FACS), magnetic-activated cell sorting (MACS) and sorting based on biotin labelling. Sorting of the cells can be achieved by direct detection of the protein encoded by the reporter gene, such as when the reporter gene encodes a fluorescent protein, or indirect, such as when the reporter gene encodes a protein to which an antibody (which could be fluorescently-labelled, biotin-labelled or labelled in any other way that allows sorting) specific for the protein binds, or magnetic beads coated with an antibody specific for the protein binds. Methods for sorting cells on the basis of expression of an intracellular protein or cell-surface protein are well known in the art and can be used herein, and it is well within the ability of a skilled person to select the appropriate sorting technique based on the nature of the reporter gene. In addition to simply sorting cells that are positive for reporter gene expression, the degree of reporter gene expression can also be used to sort cells. For example, cells can be sorted into low-, medium- and high-expressers, and cap genes recovered from one or more of those populations.

Selection based on transgene expression can be performed by simply detecting the protein expressed by the reporter gene, selecting cells in which that protein has been detected, and recovering cap genes from DNA extracted from those cells. In other embodiments, selection is performed predominantly on the basis of gene expression (cap gene or reporter gene or both) at the RNA level.

In one embodiment, selection is made on the basis of protein expression of the reporter gene alone. Replication-incompetent AAV containing a genome with ITRs flanking a reporter gene and a cap gene from the cap library are transduced into host cells. The reporter gene and a cap gene can be under the control of the same promoter (e.g. a ubiquitous or tissue-specific promoter selected on the basis that it is functional in the host cells used for transduction and under the conditions of the selection process) or different promoters. Thus, in some examples, the reporter gene is operably linked to a first promoter, and the cap gene is operably linked to a second promoter. In these examples, the first promoter may be ubiquitous or may be tissue-specific and selected on the basis that it is functional in the host cells used for transduction and under the conditions of the selection. The second promoter can be any promoter suitable for driving expression of the cap gene, although conveniently may be a natural AAV promoter, such as the p40 promoter. Typically, the reporter gene and cap gene are oriented in different directions so that transcription proceeds in different directions along the genome. Generally, the cap gene also contains a 3′UTR directly downstream, and the reporter gene may contain a polyadenylation sequence. The genome does not contain a functional rep gene, thus rendering the AAV replication-incompetent.

Following transduction of the cells with the AAV and subsequent culture under conditions to facilitate reporter gene expression, cells in which protein expression from the reporter gene is detected are selected by sorting. DNA is then extracted from these selected cells and the cap gene is recovered. Recovery of the cap gene can be performed in any manner known to those skilled in the art. Typically PCR amplification of the cap gene is performed to recover the gene, such as by using primers specific for the 5′ and 3′ ends of the cap gene, or primers specific for regions upstream and downstream of the cap gene, such as primers specific for the second promoter and the 3′ UTR that flank the cap gene.

Selection can also be performed on the basis of gene expression at the RNA level. In this embodiment, cells positive for the protein product of the reporter gene are first selected (e.g. by FACS, MACS or biotin labelling and sorting), and then RNA transcripts from the capsid and/or reporter gene are detected and assessed (e.g. by conversion to cDNA and the amplification and/or sequencing to detect and/or identify capsid and/or reporter gene sequences) so as to identify capsids that have been enriched in the selection process. Several vector designs can be utilised for these platforms, including those in which the cap and reporter genes are under the control of the same or different promoters, and optionally comprise one or more barcodes (see WO2020077411).

In one embodiment, the replication-incompetent AAV contains a genome with ITRs flanking a cap gene (e.g. from a library) and a reporter gene, where the cap gene and reporter gene are under the control of the same promoter but are separated by an internal ribosome entry site (IRES). The cap gene may be upstream or downstream of the reporter gene. In this embodiment, the target cells are transduced and cells are sorted (or selected) on the basis of reporter protein expression. RNA and optionally DNA are then extracted from the cells. The RNA is converted to cDNA and the capsid sequences are detected and recovered from the cDNA and then used to produce replication-incompetent AAV for the HR selection described herein.

In this embodiment, the promoter used to drive the expression of the cap and reporter genes is functional in the host cells used for selection and under the conditions used for selection. Typically, the promoter is constitutive in the host cells used for selection and potentially also for the downstream therapeutic applications. In particular examples, the promoter is a ubiquitous promoter (i.e. functional in multiple tissue types or multiple host cells) and/or a constitutive promoter. In other examples, the promoter is tissue-specific. Suitable promoters are well known to those skilled in the art and non-limiting examples are provided below. Recovery of the cap gene can be performed by any manner known to those skilled in the art, as described above.

In other embodiments, the replication-incompetent AAV contains a genome with ITRs flanking a reporter gene operably linked to a first promoter, and a cap gene (e.g. from a capsid library) operably linked to a second promoter. In this embodiment, the target cells are transduced and cells are sorted (or selected) on the basis of reporter protein expression. RNA and optionally DNA are then extracted from the cells. The RNA is converted to cDNA and the capsid sequences are detected and recovered from the cDNA or DNA and then used to produce replication-incompetent AAV for the HR round of selection. For this dual-promoter embodiment to be effective without the need for barcodes, the promoters used to drive the expression of the cap and reporter genes must be functional in the host cells used for selection and under the conditions used for selection. Recovery of the cap gene can be performed by any manner known to those skilled in the art, as described above.

### Nucleic Acid Molecules and Methods for Producing Replication-incompetent AAV

The replication-incompetent AAV provided herein and used in the methods of the present disclosure are produced by packaging the AAV genomes broadly described above. Because the genomes lack a rep gene, this is provided in trans for packaging, along with Adenovirus helper functions or helper viruses.

Thus provided herein are nucleic acid molecules and methods for producing replication-incompetent AAV. The AAV are produced by introducing into a cell a first nucleic acid molecule comprising an AAV genome comprising two AAV ITRs flanking left and right homology arms flanking a cap gene, optionally operably linked to a first promoter; a second nucleic acid molecule comprising a rep gene operably linked to a second promoter; and a third nucleic acid molecule comprising Adenovirus helper functions, or an helper virus; and culturing the cell under conditions suitable for packaging the genome, thereby producing replication-incompetent AAV.

An appropriate promoter for driving expression of the cap gene can be selected by the skilled person. In some examples, the promoter is ubiquitous and/or constitutive, e.g. the spleen focus forming virus (SFFV) promoter, the elongation factor-1 alpha promoter (EF-1α), the short elongation factor-1 alpha promoter (EFS), Rous sarcoma virus (RSV) LTR promoter (optionally with the RSV enhancer), the cytomegalovirus (CMV) promoter (optionally with the CMV enhancer), the SV40 promoter, the dihydrofolate reductase promoter, the β-actin promoter, or the phosphoglycerol kinase (PGK) promoter, which can be of human or non-human origin. In other examples, the promoter is tissue specific. If the latter, then the promoter may be selected based on the type of host cell that is being transduced. In further examples, the promoter that drives expression of the cap gene is an AAV promoter, such as the p5, p19 or p40 promoter.

The cap gene is from a cap library. Any AAV cap library can be used to source the cap genes, such as libraries based on shuffled DNA, error-prone PCR, or peptide display. Generally, the cap gene further contains a 3′ UTR and/or a 5′ UTR, such as a native AAV 3′ UTR or 5′ UTR as is known to those skilled in the art and routinely contained in AAV vectors.

AAV ITRs used in the nucleic acid molecules of the disclosure may have a wild-type sequence or may be altered, e.g., by the insertion, deletion or substitution of nucleotides. Additionally, AAV ITRs may be derived from any of several AAV serotypes, including without limitation, AAV1, AAV2, AAV3, AAV4, AAV5, AAV6, AAV7, AAV8, AAV9, AAV10, AAV11, AAV12 or AAV13. Such ITRs are well known in the art.

The AAV rep gene in the second nucleic acid molecule may have a wild-type nucleotide sequence or may be altered, e.g., by the insertion, deletion or substitution of nucleotides, provided it retains the ability effect AAV replication. Additionally, AAV rep may be derived from any of several AAV serotypes, including without limitation, AAV1, AAV2, AAV3, AAV4, AAV5, AAV6, AAV7, AAV8,AAV9, AAV10, AAV11, AAV12 or AAV13 and any variants thereof. In particular embodiments, the rep gene is from the same AAV serotype as the ITRs, and/or the same serotype as any AAV promoter used to drive expression of the cap gene in the first nucleic acid molecule.

As would be appreciated, a library of replication-incompetent AAV are produced when a plurality of the first, second and third nucleic acid molecules are introduced into a plurality of host cells. Thus, also provided are libraries of replication-incompetent AAV and pluralities of the first, second and third nucleic acid molecules (e.g., at least 10, 10², 10³, 10⁴, 10⁵, 10⁶, 10⁷, 10⁸, 10⁹, 10¹⁰, 10¹¹ or 10¹² AAV or first, second and third nucleic acid molecules). In these embodiments, a plurality of cap genes having two or more different nucleic acid sequences is contained in the library or plurality. Generally, at least 80%, 81%, 82%, 83%, 84%, 85%, 86%, 87%, 88%, 89%, 90%, 91%, 92%, 93%, 94%, 95%, 96%, 97%, 98% or 99% of the first nucleic acid molecules, or the AAV, comprise a cap gene with a unique nucleic acid sequence relative to the other cap genes nucleic acid molecules or AAV.

Helper viruses and helper functions for AAV are known in the art. The helper functions may be provided by one or more helper plasmids or helper viruses comprising adenoviral helper genes. Non-limiting examples of the adenoviral helper genes include E1A, E1B, E2A, E4 and VA, which can provide helper functions to AAV packaging. Helper viruses include, for example, viruses from the family Adenoviridae and the family Herpesviridae. Examples of helper viruses of AAV include, but are not limited to, SAdV-13 helper virus and SAdV-13-like helper virus described in US20110201088, helper vectors pHELP (Applied Viromics). A skilled artisan will appreciate that any helper virus or helper plasmid of AAV that can provide adequate helper function to AAV can be used herein.

Various types of cells can be used to package AAV. For example, packaging cell lines that can be used include, but are not limited to, HEK 293 cells, Hela cells, and Vero cells, for example as disclosed in US20110201088.

The nucleic acid molecules can also include transcriptional enhancers, translational signals, and transcriptional and translational termination signals. Examples of transcriptional termination signals include, but are not limited to, polyadenylation signal sequences, such as bovine growth hormone (BGH) poly (A), SV40 late poly (A), rabbit beta-globin (RBG) poly (A), thymidine kinase (TK) poly (A) sequences, and any variants thereof. In some embodiments, the transcriptional termination region is located downstream of the posttranscriptional regulatory element. In some embodiments, the transcriptional termination region is a polyadenylation signal sequence. In other embodiments, introns are included in the nucleic acid molecules, such as between a promoter and an operably linked gene.

In particular embodiments, the nucleic acid molecules are plasmids.

### AAV Vectors

Also contemplated are methods for producing AAV vectors using the cap genes identified according to methods described herein, and the resulting AAV vectors themselves. Methods for vectorizing a capsid protein are well known in the art and any suitable method can be employed for the purposes of the present disclosure. For example, the cap gene can be recovered (e.g. by PCR or digest with enzymes that cut upstream and downstream of cap) and cloned into a packaging construct containing rep. Typically, the cap gene is cloned downstream of rep so the rep p40 promoter can drive cap expression. This construct does not contain ITRs. This construct is then introduced into a packaging cell line with a second construct containing a transgene flanked by ITRs. Helper function or a helper virus are also introduced, and recombinant AAV comprising a capsid generated from capsid proteins expressed from the cap gene, and encapsidating a genome comprising the transgene flanked by the ITRs, is recovered from the supernatant of the packaging cell line. Various types of cells can be used as the packaging cell line. For example, packaging cell lines that can be used include, but are not limited to, HEK 293 cells, Hela cells, and Vero cells, for example as disclosed in US20110201088. The helper functions may be provided by one or more helper plasmids or helper viruses comprising adenoviral helper genes. Non-limiting examples of the adenoviral helper genes include E1A, E1B, E2A, E4 and VA, which can provide helper functions to AAV packaging. Helper viruses of AAV are known in the art and include, for example, viruses from the family Adenoviridae and the family Herpesviridae. Examples of helper viruses of AAV include, but are not limited to, SAdV-13 helper virus and SAdV-13-like helper virus described in US20110201088, helper vectors pHELP (Applied Viromics). A skilled artisan will appreciate that any helper virus or helper plasmid of AAV that can provide adequate helper function to AAV can be used herein.

In some instances, rAAV virions are produced using a cell line that stably expresses some of the necessary components for AAV virion production. For example, a plasmid (or multiple plasmids) comprising the nucleic acid containing a cap gene identified as described herein and a rep gene, and a selectable marker, such as a neomycin resistance gene, can be integrated into the genome of a cell (the packaging cells). The packaging cell line can then be transfected with an AAV vector and a helper plasmid or transfected with an AAV vector and co-infected with a helper virus (e.g., adenovirus providing the helper functions). The advantages of this method are that the cells are selectable and are suitable for large-scale production of the recombinant AAV. As another non-limiting example, adenovirus or baculovirus rather than plasmids can be used to introduce the nucleic acid encoding the capsid polypeptide, and optionally the rep gene, into packaging cells. As yet another non-limiting example, the AAV vector is also stably integrated into the DNA of producer cells, and the helper functions can be provided by a wild-type adenovirus to produce the recombinant AAV.

As will be appreciated by a skilled artisan, any method suitable for purifying AAV can be used in the embodiments described herein to purify the recombinant AAV, and such methods are well known in the art. For example, the recombinant AAV can be isolated and purified from packaging cells and/or the supernatant of the packaging cells. In some embodiments, the AAV is purified by separation method using a CsCl gradient. In other embodiments, AAV is purified as described in US20020136710 using a solid support that includes a matrix to which an artificial receptor or receptor-like molecule that mediates AAV attachment is immobilized.

### Compositions, Combinations and Kits

Also contemplated herein are compositions comprising any one or more of the replication-incompetent AAV, AAV vectors or nucleic acid molecules described above and herein; combinations comprising any one or more of the replication-incompetent AAV, AAV vectors or nucleic acid molecules described above and herein, and host cells comprising any one or more of the replication-incompetent AAV, AAV vectors or nucleic acid molecules described above and herein.

Also provided are kits, such as for use in the methods of the disclosure for producing replication-incompetent AAV and for selecting cap genes for vectorization. The kits therefore may include any one or more of the nucleic acid molecules described above and herein, optionally with instructions for use. Primers suitable for amplification of regions of the nucleic acid molecules may also be included in the kit.

In order that the invention may be readily understood and put into practical effect, particular preferred embodiments will now be described by way of the following non-limiting examples.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

### EXAMPLES

### Example 1. Materials and Methods

### Plasmids and Libraries

Plasmids for HR selection platform contain the AAV2 ITRs flanking a cassette containing left and right homology arms (HAs) with sequences that are homologous to the TRAC or BTK genes, where the HAs flank the p40 promoter, AAV2 intron, cap gene and polyadenylation sequence. Sequences of primers are shown in Table 2. Briefly, the p40 promoter and intron elements were amplified from plasmids described in WO2020077411 using primers VG1228/VG1229 and inserted into a HTE plasmid (see WO2020/077411) using BsiWI and SwaI restriction sites. Primers VG1234/VG1235 were used to amplify the TRAC left HA and primers B-LHA_F/B-LHA_R were used to amplify the BTK left HA, and the amplicons inserted into the plasmid using SpeI and BsiWI restriction sites. Primers VG1236/VG1237 were used to amplify the TRAC right HA and primers B-RHA_F/B-RHA_R were used to amplify the BTK right HA. The resulting amplicons were inserted using NsiI and FseI restriction sites. This resulted in a SwaI and NsiI flanked stuffer to introduce capsid libraries.

Plasmids for the FT preselection were as described in WO2020/077411 and contained AAV2 ITRs (SEQ ID NOs: 7 and 8) flanking eGFP (SEQ ID NO: 9) with a SV40 polyA sequence (SEQ ID NO: 10) under the control of the SFFV promoter (SEQ ID NO: 11) and a cap gene under the control of the p40 promoter (SEQ ID NO: 12, representing a fragment of the rep gene containing the p40 promoter). Introns between the promoters and operably linked genes were included, including the wild-type AAV2 intron between the p40 promoter and the cap gene and a SV40 intron (SEQ ID NO: 13) between the eGFP gene and the SFFV promoter. Two unique restriction sites (SwaI/NsiI) flanking the cap gene facilitate the cloning of other cap genes from AAV libraries carrying these sites.

A plasmid (p-Rep2-polyA; see WO2020077411) containing the rep gene from AAV2/2 (referred to as AAV2 for simplicity) with a polyA sequence and under the control of the endogenous p5 promoter was also utilised to produce the replication incompetent AAV.

Plasmid libraries were produced by inserting cap genes from three capsid libraries into the plasmids at the SwaI/NsiI sites. The three capsid libraries included an AAV2 peptide library (library 1), and AAV6 peptide library (library 2) and an AAV4 and AAV6 shuffled library (library 3). Plasmid library nucleic acid was amplified in competent bacteria cells and was purified using commercially available plasmid extraction kit and then packaged as essentially described below with supplementation of 7.5 μg/plate of the p-Rep2-polyA plasmid.

TABLE 2
Library construction primers

| | | SEQ |
|---|---|---|
| | | ID |
| Primer name | Primer sequence | NO |
| VG1228_p40-AAVintron_F | atgcctCGTACGACGTAGTCCCTCGCATCGC | 1 |
| VG1229_p40-AAVintron_R | atcggcagccatACCTGATTTaaatc | 2 |
| VG1234_left-HA-TRAC-F | attgactagtgccgccatggcggccgcggg | 3 |
| VG1235_left-HA-TRAC-R | agtcgtCGTACGgccgaattcagggtcaggg | 4 |
| VG1236_NsiIRHA-TRAC_F | tagtATGCATctggggagcaatcactagccag | 5 |
| VG1237_FseIRHA-TRAC-R | tatcgGGCCGGCCTtggccattcctgaagcaagg | 6 |
| B-LHA_F | TATCTACTAGTCAGAGGTGGCATCTATATCAGTAAGAC | 23 |
| B-LHA_R | TATGACGTACGGCTTCTTCTTTCTGGAGTTCACC | 24 |
| B-RHA_F | TAGTATGCATTTTCTGAAGCGATCCCAACAG | 25 |
| B-RHA_R | TAGTGGCCGGCCGAGGGATTAAGCAACCAGATGATTAG | 26 |

### AAV Production

AAV Production with Purification by Caesium Chloride Gradient

AAV libraries were packaged on 60×15 cm tissue culture dishes containing 90-95% confluent HEK 293 cells (ATCC, catalogue no. CRL-1573). Briefly, 22.5 μg of the Adenovirus 5 helper plasmid and 15 μg of the plasmid library were transfected in each of the 60 plates, by mixing the total 37.5 μg of DNA with 75 μg (ratio DNA:PEI 1:2) of polyethylenimine (PEI, MW 25000,Polysciences 23966-2) in a total volume of 500 μL of OptiMEM media (Life Technologies). 72 h post transfection cells were harvested and centrifuged at 3800 rpm for 15 min. From here media and cells were treated separately. The supernatant was re-spun and incubated 3 hours on ice with ¼ volume of 40% Polyethylene Glycol (PEG) 8000 (Fisher Scientific, BP2331-1KG). After incubation, the solution was centrifuged (3800 rpm, 4°) and PEG-pellet containing the AAV particles was resuspended in 20 mL of Cracking Buffer (10 mM Tris, pH-7.5, 0.15M NaCl, 10 mM MgCl2) over-night. On the other hand the cell pellet from the first spin was resuspended in 35 mL of Benzonase Buffer (50 mM Tris, pH 8.5, 2 mM CsCl2) and three cycles of freeze-thawing were carried out by submerging the suspension in dry ice/ethanol. After the 3rd thaw Benzonase (EMD Chemicals, Merck, 1.01695.0002) was added at a concentration of 200 U/mL and solution was incubated one hour at 37° C. Cell suspension was then centrifuged to discard cell debris. Supernatant was moved to a new 50 mL Falcon and 1/39th volume of 1M CaCl2 was added and kept on ice for one hour. The solution was then centrifuged again and supernatant was transferred to a new tube and incubated 3 hours on ice with ¼ volume of 40% PEG 8000 and spun at 3800 rpm 4° C. and pellet containing AAV particles was resuspended in 20 mL of Resuspension Buffer (50 mM Hepes, pH 7.4, 0.15M NaCl, 25 mM EDTA) over-night. Cesium Chloride (CsCl, Ultrapure optical grade, Life Technologies, 15507-023) gradient centrifugation was then carried out for the media and cell suspensions independently. Briefly, 12 mL of 1.3 g/mL CsCl in Dulbecco's Phosphate Buffered Saline (Sigma Aldrich, D8537) were added to Ultra-Clear Centrifuge Tube (Beckman Coulter, 344058). 5 mL of 1.5 g/mL CsCl in PBS were added then to the bottom of the tube to establish a clear interface. The 20 mL of the cell and media vector solutions were then added to the top of the gradient and both fractions were spun in a SW32 Ti Rotor at 25K (106,800 g) at 20° C. for 24 hours. Vector fractions laying in between the two distinct density solutions were then collected using a 10 mL syringe/18G needle. Both fractions were pulled together at this stage and a second ultracentrifugation in a CsCl solution (1.37 g/mL) was then carried out. 12 mL of the CsCl solution were added in a centrifuge tube (BeckmanCoulter, 331372) and spun at 38K in a SW41 Ti rotor (247,600 RCF) for 24 hours. 0.5 mL fractions were collected from the second spin and fractions with highest amounts of AAV DNA were determined by quantitative polymerase chain reaction (qPCR). The top six fractions were then pulled and dialyzed in order to eliminate the excess of CsCl. The AAV solution was loaded into a slide-a-lyzer dialysis cassette (10.000 MWCO, 3.0 mL capacity, Thermo Fisher, 87730) and two initial dialyses were carried out for 33 24 hours in 2 L of PBS at 4° C. A 3rd additional dialysis was then carried out in 5% Sorbitol in PBS for 3 hours at 4° C. AAV particles were then removed from the dialysis cassette and filtered through a 0.22 μm syringe filter (Merck Millipore, SLGP033RS). Finally, the 3 mL of vector solution were concentrated to 1 mL with a Amicon Ultra-15 Centrifugal Unit (NMWL of 100 kDa, Merck Millipore, UFC910024).

AAV Production with Purification by Iodixanol Gradient

Indicated AAV libraries and AAV preparations were packaged on 5×15 cm tissue culture dishes containing 90-95% confluent HEK 293 cells (ATCC, catalogue no. CRL-1573). Briefly, 22.5 μg of the Adenovirus 5 helper plasmid and 15 μg of the AAV plasmid library were transfected in each of the 5 plates, by mixing the total 37.5 μg of DNA with 75 μg (ratio DNA:PEI 1:2) of polyethylenimine (PEI, MW 25000, Polysciences 23966-2) in a total volume of 500 μl of OptiMEM media (Life Technologies) per plate. 72 h post transfection cells were harvested and centrifuged at 3800 rpm for 15 min. From here media and cells were treated separately. On one hand supernatant was re-spun and incubated 3 hours on ice with ¼ volume of 40% Polyethylene Glycol (PEG) 8000 (Fisher Scientific, BP2331-1KG). After incubation, the solution was centrifuged (3800 rpm, 4°) and PEG-pellet containing the AAV particles was resuspended in 2 mL of PBS Buffer (pH 8.0). Separately, cell pellet was resuspended in 3 mL of PBS buffer (pH 8.5) and cells were lysed using 3 freeze/thaw cycles by placing the solution at −80° C. (dry ice+ethanol) and back at 37° C. (water bath). The resuspended PEG pellet and the cell lysate were then mixed and incubated with Benzonase (200 U/mL) 1 h at 37° C. 10% sodium deoxycholate was added to a final concentration of 0.5% and subsequently ¼ volume of 5M NaCl to a final concentration of 1M. Solution was incubated for 30 minutes at 37° C. (water bath) and spun 30 minutes at 3800 rpm at 4° C. Virus supernatant was collected into a 5 mL Eppendorf tube.

The four solutions required for iodixanol gradient (15%, 25%, 40% and 60%) were prepared. The 15% solution was added to the bottom of the Beckman tube using a 10 ml syringe with a long 18-gauge metal cannula. Subsequent layers were added below the previous layer (25%->6 mL; 40%->5 mL; 60%->5 mL) by extending the syringe needle to the bottom of the Beckman tube and slowly injecting.

The recovered virus supernatant was then carefully added to the top of the gradient by slowly dripping the solution using a 5 mL pipette and avoiding disruption of the gradient layers. The remaining void of the Beckman tube was filled with balancing buffer and tubes were balanced within 0.01 g. The vector preparation(s) were centrifuged at 58.000 rpm in a Beckman Type 70Ti rotor for 2 hours and 10 minutes at 18° C. using a Beckman Coulter XPN ultracentrifuge set a 3 for acceleration and at 9 (slowest) for deceleration. After centrifugation, tubes were carefully removed and mounted on a ring stand with a utility clamp back to the tissue culture hood. Tube surface was cleaned with 70% ethanol and a 10 ml syringe with an 18-gauge needle was prepared for viral extraction upon accurate insertion approximately 1.5 mm below the interface between the 40% and 60% gradient buffer layers, with the bevel of the needle facing up. 3 to 5 mL of solution were then extracted, first with the bevelled needle opening facing upwards and then facing downward, avoiding the collection of the visible protein-rich band at the 25/40% interface.

The 3-5 mL virus preparation were then filtered with a 0.22 μm PES filter and mixed with Iodixanol Dialysis Buffer, to a total volume of 15 mL. Total volume was then moved to a 100K Amicon Ultra-4 Centrifuge Filter tube and centrifuged at 3800 rpm at 18° C. for 2-6 minutes to bring the volume down to desired volume, usually 200 to 500 μl. 15 mL of Iodixanol Dyalisis Buffer were then added and centrifuged again. This step was repeated two more times and final 200-500 μl of vector preparation were extracted from the Amicon Tube to a large cryovial tube and stored at 4° C. for short-term storage or at −80° C. for long-term storage.

### Vector Titration

qPCR was performed using standard protocols. Dilutions of 1/10 and 1/100 were used for media, and dilutions of 1/100 and 1/1000 were used for cell lysates. Primers used included SC010and SC011 (targeting GFP). Cycle: 98° C. 2 min, 39 times 98° C. 5 s+60° C. 15 s, 65° C. 30 s, melting curve from 65° C. to 95° C. by adding 0.5° C. each 5 s. Titers were averaged on the 6 measures done for each sample (2 dilutions×3 replicates) and the lysate titer was added to the media titer to obtain total titer.

### RNA Extraction and Reverse Transcription (RT)

RNA was extracted from pelleted cells following Zymoclean's direct-zol RNA microprep protocol. To further purify RNA, 3 μg of RNA were incubated 3 h at 37° C. with 4 units of NEB's Dnase1 #M03003L and NEB's DNase1 buffer #B0303S 10× (20 μL total). DNase was heat-inactivated 15 min at 70° C. Random hexamers 10× and dNTP (to a final concentration of 1 mM) were added to the RNA solution 5 min at 65° C. in order to anneal primers to RNA, then the solution was kept on ice and split in two samples for RT (incubation 10 min 53° C. than 10 min 80° C.). Sample RT+: for 20 μL, add SSIV buffer 5×, 2 μL DTT, 2 μL Dnase inhibitor and 2 μL superscript RT. Sample RT−: for 10 μL, add SSIV buffer 5×, 1 μL DTT, 1 μL Dnase inhibitor and 1 μL superscript RT. 1/16th volume of E. coli RnaseH was added and the solution was incubated 20 min at 37° C. Primer annealing, RT and RNA digestion were performed with Invitrogen's SuperScript IV reagents.

### Ribonucleoprotein Complexes

The RNP complexes for targeting of the TRAC locus contained a TRAC gRNA (5′-A*G*A*GUCUCUCAGCUGGUACAGUUUUAGAGCUAGAAAUAGCAAGUUAAAAUAAGGCUAGUCCGUUAUCAACU UGAAAAAGUGGCACCGAGUCGGUGCU*U*U*U-3′ (SEQ ID NO:14), where the asterisk (*) represents 2′-O-methyl 3′ phosphorothioate (Synthego), and SpCas9 (IDT, Alt-R HiFi).

The RNP complexes for targeting of the BTK locus contained a BTK gRNA (5′-G*A*U*GCUCUCCAGAAUCACUGGUUUUAGAGCUAGAAAUAGCAAGUUAAAAUAAGGCUAGUCCGUUAUCAACUUG AAAAAGUGGCACCGAGUCGGUGCU*U*U*U-3′ (SEQ ID NO:21), where the asterisk (*) represents 2′-O-methyl 3′ phosphorothioate (Synthego), and SpCas9 (IDT, Alt-R HiFi).

### Genomic DNA Extraction

Two different methods to extract genomic DNA from T cells were used: a phenol-chloroform extraction (Westhaus et al., 2020, doi: 10.1089/hum.2019.264) and NEB HMW kit extraction (Cat #: T3050S).

### Example 2. Selection of Capsids on the Basis of Homologous Recombination in T Cells and Hematopoietic Stem Cells

A study to assess the feasibility of selecting capsids on the basis of their ability to support homologous recombination in T cells was performed. The study was designed to have an initial preselection process in which a capsid library was first screened using the FT platform (described in WO2020077411) to select for capsids that could facilitate functional transduction of T cells (i.e. transduction of, and transgene expression in, T cells). Selection using the HR platform was then performed, where capsids were selected on the basis of their ability to support homologous recombination in T cells. FIG. 2 is a schematic outlining this process. In the process exemplified in FIG. 2, the capsid library is an AAV2 or AAV6 peptide display capsid library.

For the first round (or preselection) process using the FT platform, T cells were transduced with three different libraries (AAV2 peptide library, AAV6 peptide library, AAV4/AAV6shuffled library) packaged in the FT platform. The T cells were provided as buffy coat from the Australian Red Cross and isolated using standard CD3 MACS isolation. T cells were expanded in serum-free media and IL-6 supplement. Transduction of the libraries was performed at three different doses. Cells were harvested three days after transduction and RNA was extracted using TRIZOL precipitation (Westhaus et al., 2020, doi: 10.1089/hum.2019.264). cDNA was generated using Superscript IV first-strand synthesis system (Cat #: 18091050). The AAV2 and AAV6 peptide regions were amplified using a forward primer (CTAACCCTGTGGCCACGG; SEQ ID NO: 15) and reverse primer (CGTCTCTGTCTTGCCACACC; SEQ ID NO: 16) primer to create the PCR peptide pool. The peptide pool was subsequently cloned into the background capsids in the TRAC (FIG. 2, round 2b) or BTK (FIG. 2, round 2c) HR platforms or the FT platform (FIG. 2, round 2a). Full-length shuffled capsids were amplified using a forward primer (ATTGGCTCGAGGACAACCTCTC; SEQ ID NO: 17) and a reverse primer mix (TTAAACGGTTTATTGATTAACAGGCAATTACAGGG; SEQ ID NO: 18/TTAAACGGTTTATTGATTAACAAGCAATTACAGGTG; SEQ ID NO: 19) binding both AAV4 and AAV6 sequences. The full-length sequences were inserted into a transfer plasmid before being excised and inserted into the TRAC (FIG. 2, round 2b) or BTK (FIG. 2, round 2c) HR platforms or the FT platform (FIG. 2, round 2a).

For the second round (2a) process using the FT platform, T cells were transduced with three different libraries (AAV2 peptide library, AAV6 peptide library, AAV4/AAV6 shuffled library)

packaged in the FT platform (as described in [00104]). Transduction of the libraries was performed at three different doses. Cells were harvested three days after transduction and RNA was extracted using TRIZOL precipitation (Westhaus et al., 2020, doi: 10.1089/hum.2019.264). cDNA was generated using Superscript IV first-strand synthesis system (Cat #: 18091050). The AAV2 and AAV6 peptide regions were amplified using a forward primer (CTAACCCTGTGGCCACGG; SEQ ID NO:15) and reverse primer (CGTCTCTGTCTTGCCACACC; SEQ ID NO: 16) primer to create the secondary (2a) PCR peptide pool. The pool was submitted for NGS analysis.

For the second round (2b) process using the TRAC HR platform, T cells were electroporated with sgRNA/Cas9 RNP complexes before being transduced with the three pre-selected libraries. Transduction occurred either 15 min, 2 hours or 4 hours after electroporation of the T cells. Following the transduction, the cells were expanded in IL6-containing media for 14 days before DNA extraction and recovery of peptide variants using in and out PCR (TRAC genomic fwd primer: GCCAGAGTTATATTGCTGGGGTTTTG (SEQ ID NO:20) and AAV peptide reverse primer: CGTCTCTGTCTTGCCACACC (SEQ ID NO:16)). Full-lengths capsids were recovered using the same TRAC forward primer and the reverse primer mix (TTAAACGGTTTATTGATTAACAGGCAATTACAGGG (SEQ ID NO:18)/TTAAACGGTTTATTGATTAACAAGCAATTACAGGTG (SEQ ID NO:19)) binding both AAV4 and AAV6 sequences. The peptide amplicon was further processed by cleavage using the MscI enzyme yielding a short DNA fragment compatible with NGS. The full lengths capsids were cleaved with SwaI allowing integration into a transfer plasmid and analysis of colonies to identify novel sequences by Sanger sequencing.

For the second round (2c) process using the BTK HR platform, hematopoietic stem cells (HSCs) (from apharesis) were electroporated with sgRNA/Cas9 RNP complexes before being transduced with the three pre-selected libraries (1a in T cells). Transduction occurred 15 min after electroporation of the HSCs. Following the transduction, the cells were expanded in stem cell media for 14 days before DNA extraction and recovery of peptide variants using in and out PCR (using a BTK genomic fwd primer (GGAGTATTGTAGGTTTGGGGAGGC (SEQ ID NO:22)) and a AAV peptide reverse primer: CGTCTCTGTCTTGCCACACC (SEQ ID NO: 16)). Full-lengths capsids were recovered using the same BTK forward primer and the reverse primer mix

(TTAAACGGTTTATTGATTAACAGGCAATTACAGGG (SEQ ID NO:18)/TTAAACGGTTTATTGATTAACAAGCAATTACAGGTG (SEQ ID NO: 19)) binding both AAV4 and AAV6 sequences. The peptide amplicon was further processed by cleavage using the MscI enzyme yielding a short DNA fragment compatible with NGS. The full lengths capsids were cleaved with SwaI allowing integration into a transfer plasmid and analysis of colonies to identify novel sequences by Sanger sequencing. The functionality of the libraries was measured as an integration efficiency into the BTK locus using a copy number analysis of the integrated fragment by in and out ddPCR.

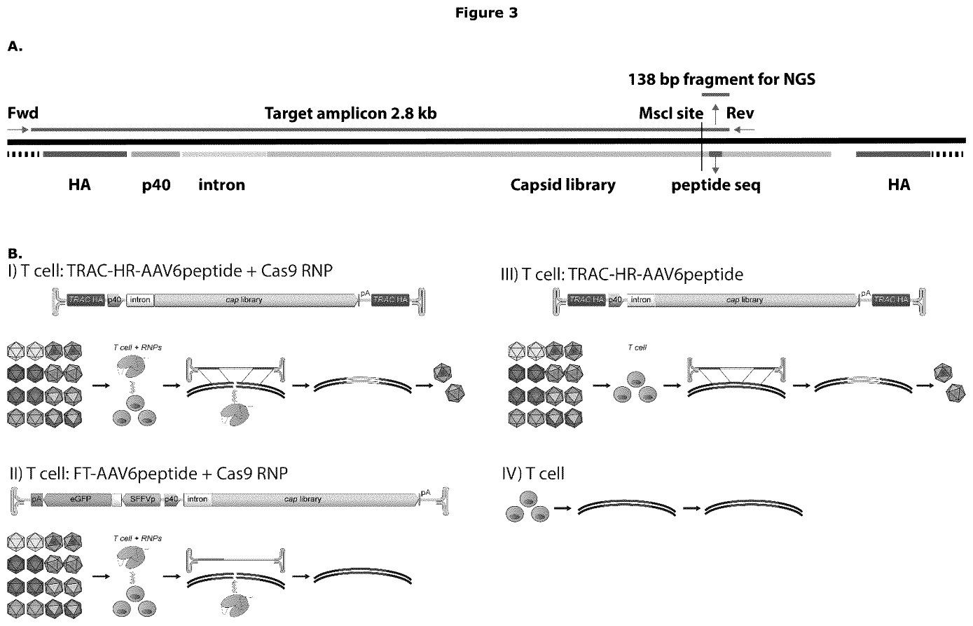

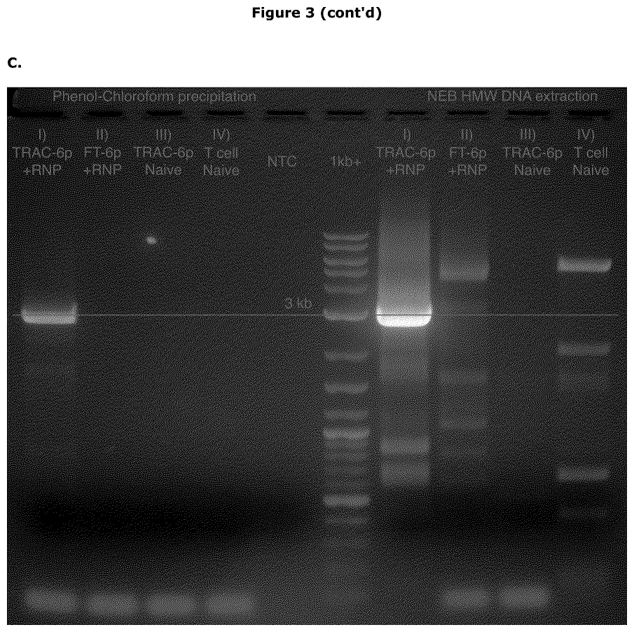

The process was initially validated using the AAV6 peptide display capsid library with a series of control conditions. FIG. 3 provides the PCR recovery method used for the peptide display capsid genes that have integrated (i.e. for the HR platform selection) and the strategy used to validate specificity of the PCR. The forward primer (GCCAGAGTTATATTGCTGGGGTTTTG; SEQ ID NO: 20) binds to a sequence in the genome upstream of the left HA (in this case, a sequence in the TRAC gene), while the reverse primer (CGTCTCTGTCTTGCCACACC; SEQ ID NO: 16) binds after the peptide coding region. For the AAV6 peptide display capsid library (as with the AAV2 peptide display capsid library), the expected length of this amplicon is 2.8 kb. This fragment can be further digested with blunt cutting restriction enzymes such as MscI to create a short fragment covering the peptide region which can be submitted for sequencing (e.g. PE150 Illumina sequencing).

For validation of the HR platform selection process, and as shown in FIG. 3B, a series of conditions were used: I. SpCas9 RNP-electroporated T cells transduced with a pre-selected (i.e. using the FT preselection round described above) TRAC-HR. AAV6 peptide display library; II. SpCas9 RNP-electroporated T cells transduced with a pre-selected FT.AAV6 peptide display library serving as a negative control (as targeted integration of the library was not expected); (III) Untreated T cells transduced with a pre-selected TRAC-HR.AAV6 peptide display library serving as a control to test if the SpCas9-mediated DNA break is necessary for selection; and (IV) Untreated control T cells serving as an additional negative control. As shown in FIG. 3C, a band at the expected size of about 2.8 kb was detected only in SpCas9 RNP-electroporated T cells transduced with the pre-selected TRAC-HR.AAV6 peptide display library. No band was detected in any of the control conditions. This confirmed that the HR selection platform operated as designed.

### Example 3. Use of the HR Selection Platform in T Cells with Three Libraries

### Selection of Capsid Libraries

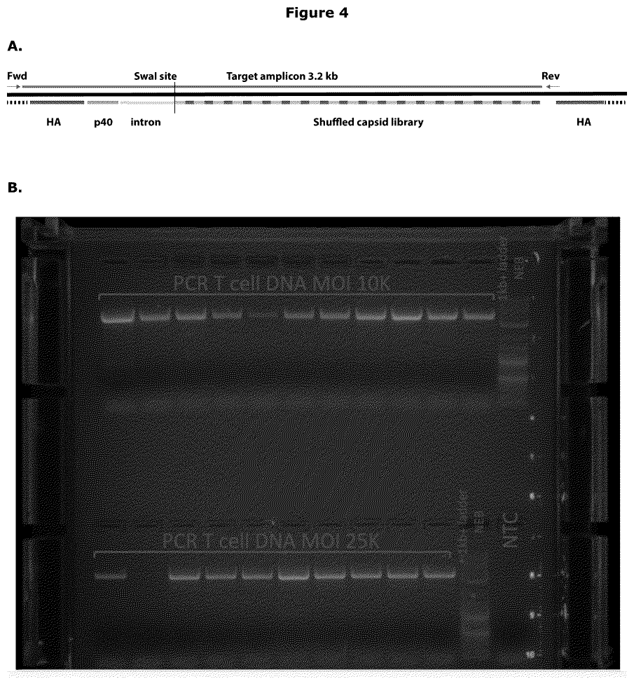

Three capsid libraries were screened in T cells using the two-round selection process described in Example 2: an AAV2 peptide display capsid library, an AAV6 peptide display capsid library, and an AAV4/AAV6 shuffled capsid library. For the FT preselection of the AAV4/AAV6 shuffled capsid library, the entire capsid gene was amplified from GFP-positive cells and cloned by Gibson assembly into a transfer plasmid followed by transfer into the HR platform (using SwaI and NsiI restriction) to produce the library for the second round HR-based selection. HR-mediated integration of the capsid gene in the second round was detected using the same forward primer as used for the peptide display library and a reverse primer mix (TTAAACGGTTTATTGATTAACAGGCAATTACAGGG (SEQ ID NO:18)/TTAAACGGTTTATTGATTAACAAGCAATTACAGGTG (SEQ ID NO:19)) binding both AAV4 and AAV6 sequences, resulting in an amplicon of 3.2 kb, and the entire capsid gene was then sequenced (see FIG. 4). The peptide libraries were selected as described in Example 2.

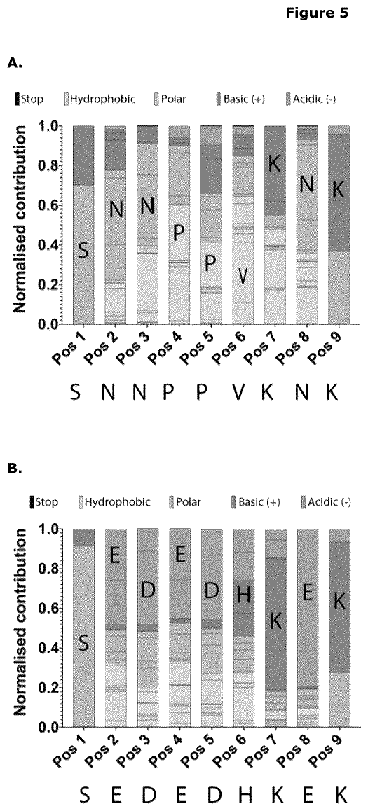

As shown in FIG. 5, the application of the HR selection platform on the AAV2 and AAV6peptide display libraries resulted in position-specific amino acid enrichment that was distinct for each library, indicating that the selection platform was selecting for variants with particular sequence preferences specific for each parent capsid. Similarly, the AAV4/AAV6 shuffled capsid library was also enriched for sequences and modifications at particular regions of the capsid (data not shown).

### Initial Individual Testing of Novel AAV Variants

A number of selected capsid genes from each library were then used to produce individual AAV variants for further assessment. These AAV variants comprise the selected variant capsids with a packaged genome containing the ITRs flanking a left and right TRAC HA either side of a promoterless GFP, enabling GFP expression following successful integration into the TRAC locus (using the endogenous TRAC promoter). The AAV variants were tested for their ability to support integration of the promoterless GFP into the TRAC locus, along with AAV6, AAV2, AAV4, AAV12, AAV4/AAV6 hybrids and AAV12/AAV6 hybrids (Viney et al., 2021, J Virol 95 (7): e02023-20), and AAV6 triple mutant (AAV6.TM; Ling et al., 2016; Sci. Rep 6:35495). Expansion of the T cells (and thus toxicity of the AAV) was also assessed. Briefly, T-cells were electroporated using the SpCas9/gRNA RNP complexes, followed by individual AAV transduction at a dose of 10,000 vg/cell. Levels of homology directed repair (HDR) were evaluated by flow cytometry 14 days after treatment. The number of T cells were also counted to assess expansion and thus determine potential toxicity from each AAV. Comparisons of GFP HDR from AAV6 with other controls revealed that apart from a previously published AAV6 triple mutant (AAV6.TM) no currently published T-cell AAV caused higher HDR than AAV6. Therefore, AAV6 was used as the benchmark and all GFP expression (Y-axis) was adjusted to it. The strongest expansion of T-cells was observed after transduction of ‘No AAV’ and AAV2, AAV4 & AAV12, hence the ‘No AAV’ control was used to adjust the expansion values (X-axis).

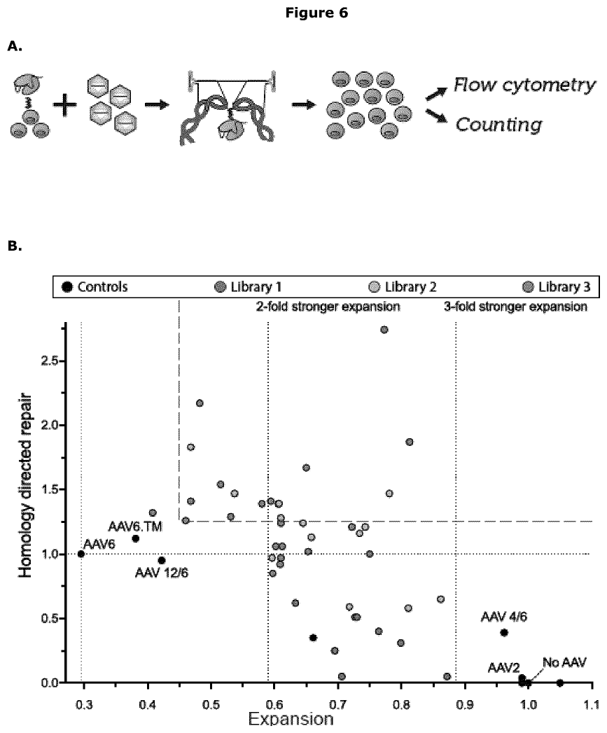

As shown in FIG. 6B, AAV6 was by far the most toxic AAV, causing the cells to expand the least. Novel variants recovered by directed evolution from Library 1 were much less toxic, but were mostly far worse at HDR than AAV6. Novel variants recovered from Library 2 and Library 3 were also less toxic than AAV6. Additionally, many of them mediated a higher levels of HDR than AAV6.

### Example 4. Utility of Different Platforms with the Same Synthesized Library

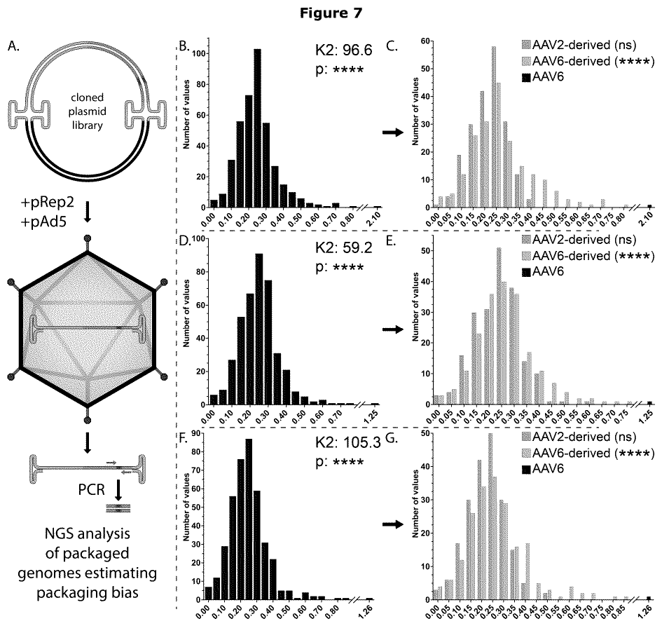

The same capsid libraries (AAV2 and AAV6 peptide libraries produced using synthetic oligonucleotides [custom order, Twist Biosciences]) were used for selection using three different platforms: the FT platform, the TRAC HR platform and the BTK HR platform. The sequences for the synthetic libraries were derived from round 2 of the FT (2a) and TRAC HR (2b) selection. The top candidates from the AAV2 peptide and AAV6 peptide libraries from both selection methods were determined using a custom bio-informatics NGS analysis pipeline. 400 peptides were chosen and each was coded in duplicates using an alternative codon version of the peptide. Local-codon-optimised AAV6 in the respective selection platforms was manually spiked in the plasmid pool to include an external benchmark. FIG. 7 shows the successful construction of the synthetic oligonucleotide libraries after vector production. A high level of similarity was achieved in library construction between all platforms ensuring that the experimental analysis will evaluate the different selection pressures applied by the platform designs rather than other biases. A single round of selection using the FT platform and the TRAC HR platform was performed in T cells side-by-side using the methods described in Example 2. Selection using the BTK HR platform will be performed in HSCs in essentially the same manner as for the TRAC HR platform.

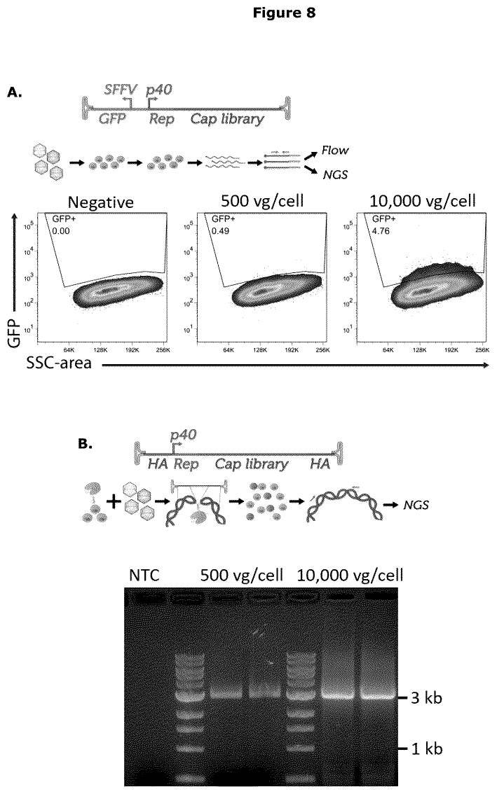

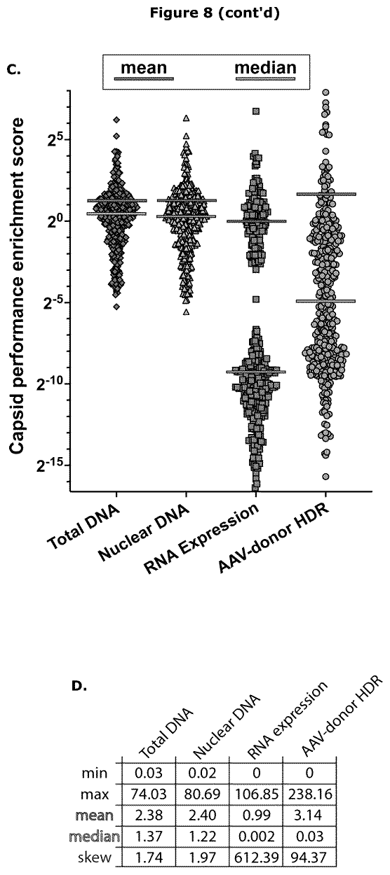

As shown in FIGS. 8 A and B, GFP expression was observed following FT library transduction into primary human T cells, and the expected 3.2 kb amplicon was detected following PCR rescue of the integrated capsid genes from gene edited primary human T cells following HR library transduction, demonstrating that the synthetic oligonucleotide libraries were functional.

FIGS. 8 C and D show that the selection based on homology-directed repair (HDR, TRAC-HR platform) was more efficient and stringent than using total or nuclear DNA and similarly stringent as RNA-based recovery using the FT-platform.

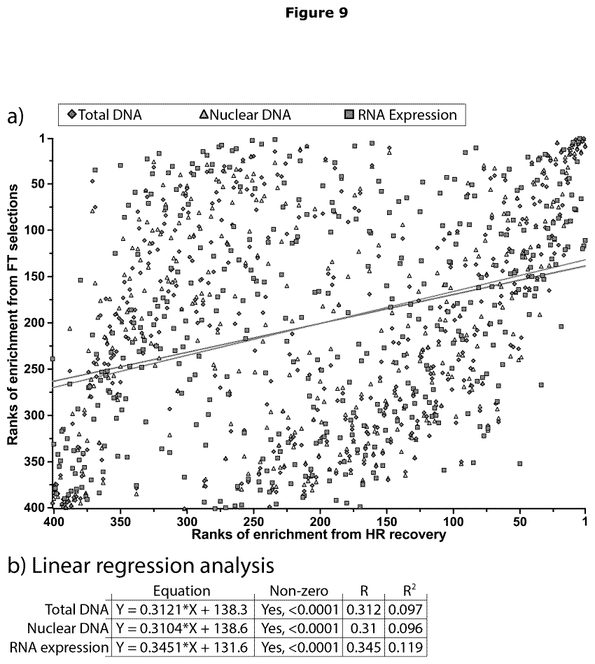

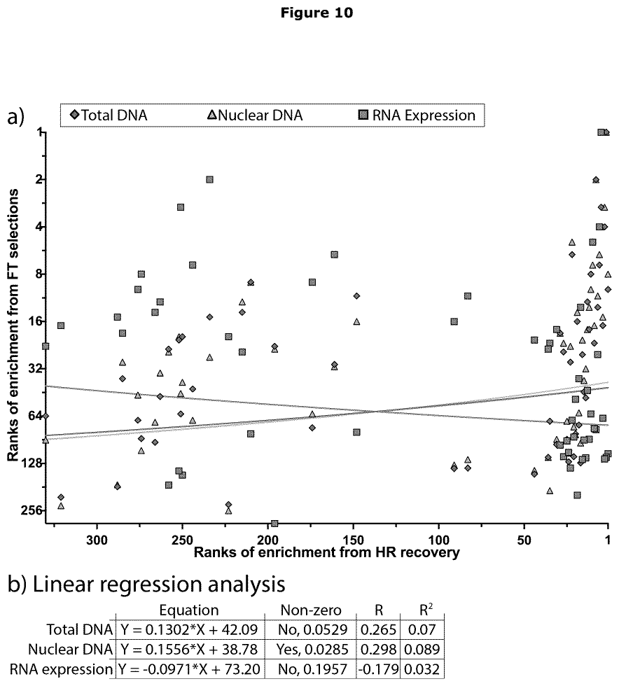

FIG. 9 shows how the functionality of the 400 capsids based on the DNA/nuclear DNA and RNA (three FT-recoveries) compared to the HDR efficiency (TRAC-HR recovery). Considering all 400 candidates, there appeared to be a high correlation between all FT-recovery techniques and the TRAC-HR platform. Considering only the top candidates (FIG. 10) it became apparent that the functionality based on the RNA expression does not correlate with the functionality on the HDR efficiency, proving that the TRAC-HR platform selection technology was vital to select novel capsids with high efficiency for HDR.

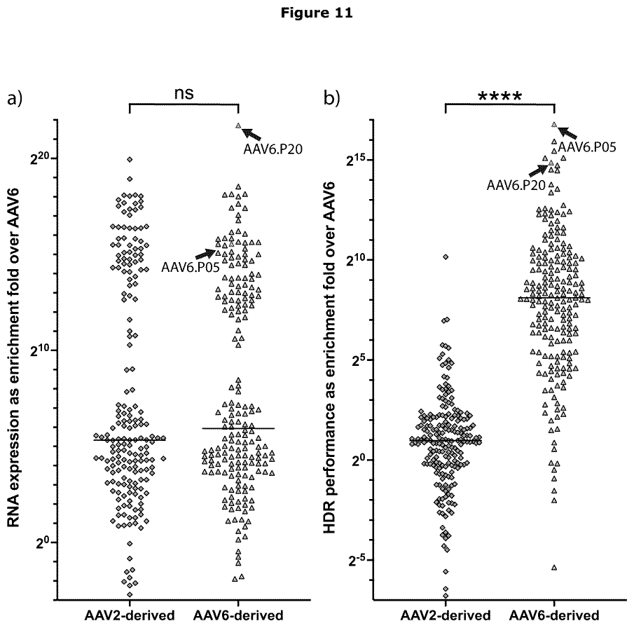

Comparing the top candidates from the FT-RNA recovery and the HDR recovery (FIG. 11), it was confirmed that the top variant for HDR (AAV6.P05) was not in the top candidates of the FT-RNA recovery. Furthermore, all AAV2-based variants performed much better with the FT-RNA method than the TRAC-HR method, confirming again that the HR selection is vital to generate novel capsids for high HR efficiencies.

### Multiplexed High-throughput Parallel Testing of Novel AAV

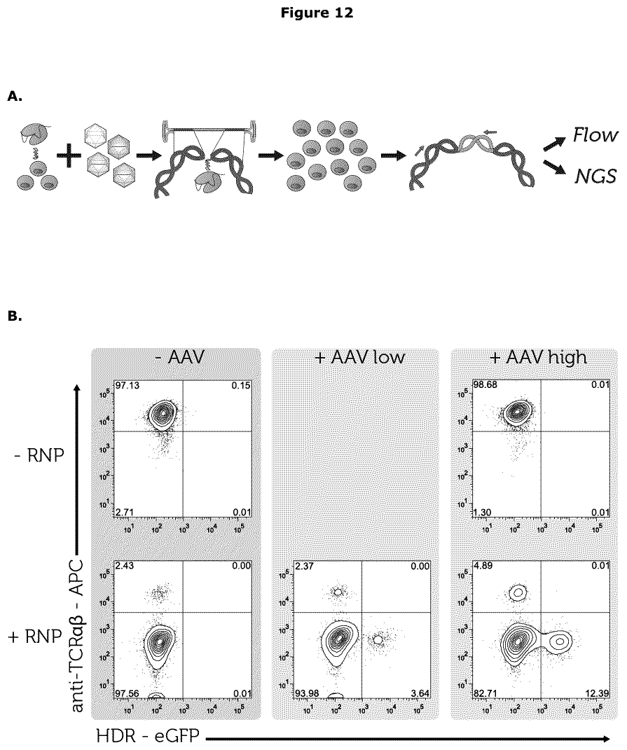

Multiplexed high-throughput parallel testing was then performed on 24 novel AAV capsid variants and an AAV6 capsid packaged to containing a barcoded promoterless GFP flanked by TRAC HAs. An equimolar mix of these 25 AAV was created which is compatible with high-throughput parallel analysis of AAV entry (PCR from total DNA of T-cells that were transduced with the AAV mix, but not electroporated with RNPs) and AAV-mediated HDR by performing a PCR with primer binding sites inside the transgene and outside the HAs. In addition to NGS analysis, cells were flow analysed for GFP expression to evaluate the overall functionality of the AAV mix, as well as allele targeting by the RNP shown as a reduction of TCR alpha-detection by APC-labelled antibodies. The results are shown in FIG. 12.

The first important metric was the efficient cutting of the TRAC locus by the RNP electroporation. As expected, mock electroporated T-cells (−RNP/−AAV) showed that 97% of T-cells were TCR alpha-positive. Following RNP treatment (+RNP/−AAV) the percentage of TCR alpha-positive cells dropped to 2% indicating a functional cutting efficiency of around 95%. Transducing the edited cells with the AAV mix at a dose of 500 vg/cell (+RNP/+AAV low), the percentage of GFP positive cells was 4%. The percentage of GFP positive cells was greatly increased by applying a higher dose of AAV (+RNP/+AAV high); up to around 12%.

Taken together, these results indicated that the AAV mix used in this study was able to mediate functional HDR at levels readily detectable by flow cytometry, giving high confidence in the ability to recover integrated NGS barcodes by PCR.

Of note, substantial GFP expression was not observed in cells that were transduced with AAV but not electroporated with RNPs (−RNP/+AAV high). This further validates that the detected GFP did indeed originate from precisely integrated AAV transgene templates.

Novel AAV Performance when Transduced as Competitive NGS Mix

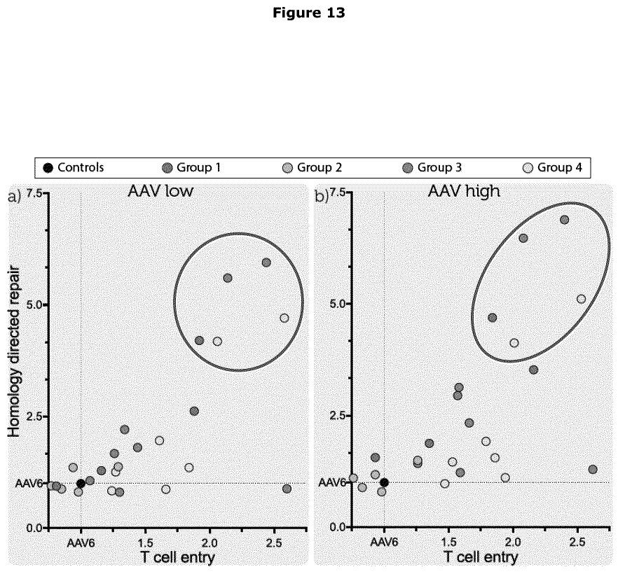

The novel variants were grouped by their capsid modifications and assessed (FIG. 13). Data for T-cell entry (X-axis) and AAV mediated HDR (Y-axis) was adjusted to the performance of AAV6. Overall, AAV6 was one of the worst performing candidates and was substantially outperformed by most novel variants. Of note, while efficient entry of AAVs into T-cells was a requirement for efficient gene editing, a high entry did not guarantee high gene editing efficiencies for all conditions tested. It was observed that a low dose of the AAV NGS mix allowed a variant from Group 4 to mediate the strongest HDR, while a high dose of the AAV NGS mix enabled stronger HDR from two AAVs of Group 1 and Group 3.

Collectively, the results demonstrate that the HR selection platform is effective in selecting and identifying novel AAV capsid variants that support efficient HR-mediated gene editing, with improved performance compared to AAV6.

As the top performers in FIG. 13 correspond to the top performer in FIG. 11 (containing AAV6.P05) these results validate the data generated with the synthetic oligonucleotide-based libraries.

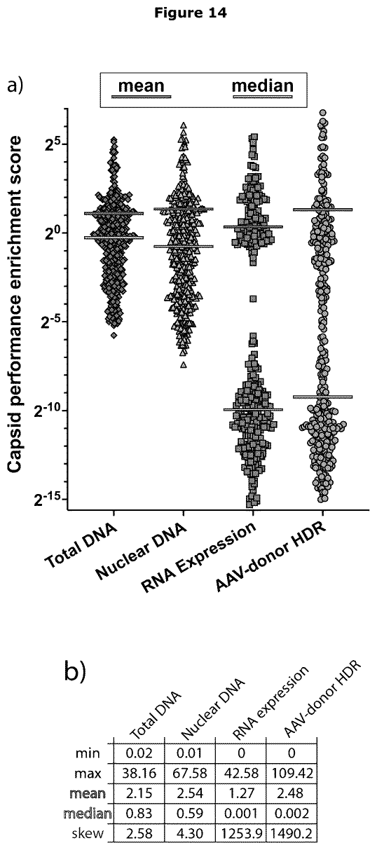

FIG. 14 shows that the selection based on homology-directed repair (HDR, BTK-HR platform) was more efficient and stringent than using total or nuclear DNA and similarly stringent as RNA-based recovery using the FT-platform.

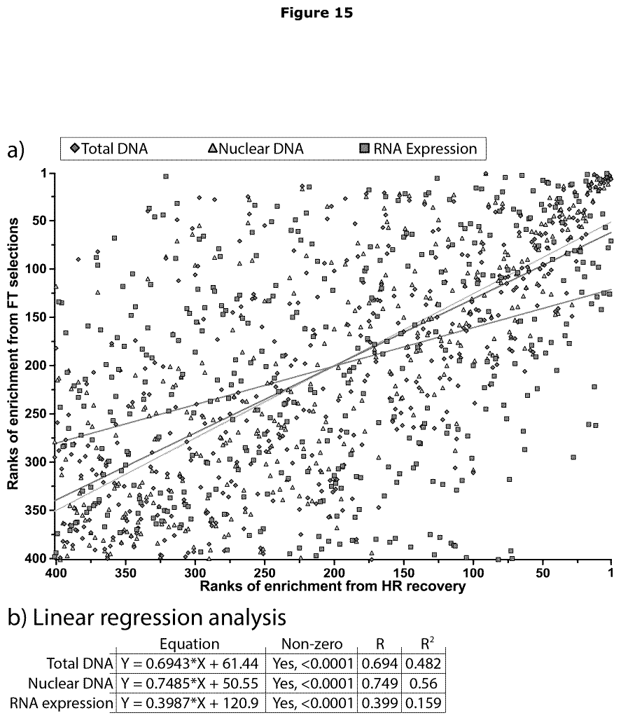

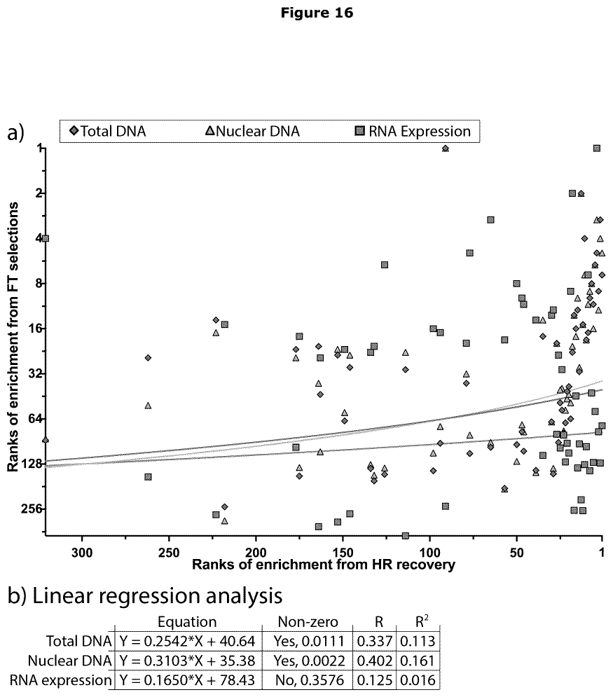

FIG. 15 shows how the functionality of the 400 capsids based on the DNA/nuclear DNA and RNA (three FT-recoveries) compared to the HDR efficiency (BTK-HR recovery). Considering all 400 candidates, there appeared to be a high correlation between the DNA FT-recovery techniques and the TRAC-HR platform, while the RNA recovery had a lower correlation with high HDR efficiency. Considering only the top candidates (FIG. 16) it became apparent that the functionality based on the RNA expression does not correlate with the functionality on the HDR efficiency, proving that the BTK-HR platform selection technology was vital to select novel capsids with high efficiency for HDR.

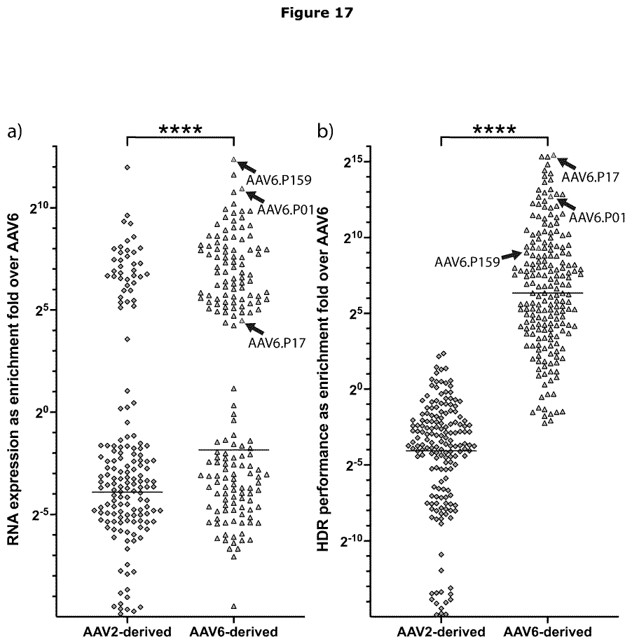

Comparing the top candidates from the FT-RNA recovery and the HDR recovery (FIG. 17), it was confirmed that the top variant for HDR (AAV6.P17) was not in the top candidates of the FT-RNA recovery, confirming again that the HR selection is vital to generate novel capsids for high HR efficiencies.

### Sequences Disclosed Herein

| SEQ ID | |
|---|---|
| NO. | Description and sequence |
| 1 | VG1228_p40-AAVintron_F: |
| | ATGCCTCGTACGACGTAGTCCCTCGCATCGC |
| 2 | VG1229_p40-AAVintron_R: |
| | ATCGGCAGCCATACCTGATTTAAATC |
| 3 | VG1234_left-HA-TRAC-F: |
| | ATTGACTAGTGCCGCCATGGCGGCCGCGGG |
| 4 | VG1235_left-HA-TRAC-R: |
| | AGTCGTCGTACGGCCGAATTCAGGGTCAGGG |
| 5 | VG1236_NsiIRHA-TRAC_F: |
| | TAGTATGCATCTGGGGAGCAATCACTAGCCAG |
| 6 | VG1237_FseIRHA-TRAC-R: |
| | TATCGGGCCGGCCTTGGCCATTCCTGAAGCAAGG |
| 7 | AAV ITR left: |
| | TTGGCCACTCCCTCTCTGCGCGCTCGCTCGCTCACTGAGGCCGGGCGACCAAAGGTC |
| | GCCCGACGCCCGGGCTTTGCCCGGGCGGCCTCAGTGAGCGAGCGAGCGCGCAGAGA |
| | GGGAGTGGCCAACTCCATCACTAGGGGTTCCT |
| 8 | AAV ITR right: |
| | AGGAACCCCTAGTGATGGAGTTGGCCACTCCCTCTCTGCGCGCTCGCTCGCTCACTGA |
| | GGCCGCCCGGGCAAAGCCCGGGCGTCGGGCGACCTTTGGTCGCCCGGCCTCAGTGA |
| | GCGAGCGAGCGCGCAGAGAGGGAGTGGCCAA |
| 9 | eGFP: |
| | TTACTTGTACAGCTCGTCCATGCCGAGAGTGATCCCGGCGGCGGTCACGAACTCCAG |
| | CAGGACCATGTGATCGCGCTTCTCGTTGGGGTCTTTGCTCAGGGCGGACTGGGTGCT |
| | CAGGTAGTGGTTGTCGGGCAGCAGCACGGGGCCGTCGCCGATGGGGGTGTTCTGCT |
| | GGTAGTGGTCGGCGAGCTGCACGCTGCCGTCCTCGATGTTGTGGCGGATCTTGAAGT |
| | TCACCTTGATGCCGTTCTTCTGCTTGTCGGCCATGATATAGACGTTGTGGCTGTTGTA |
| | GTTGTACTCCAGCTTGTGCCCCAGGATGTTGCCGTCCTCCTTGAAGTCGATGCCCTTC |
| | AGCTCGATGCGGTTCACCAGGGTGTCGCCCTCGAACTTCACCTCGGCGCGGGTCTTG |
| | TAGTTGCCGTCGTCCTTGAAGAAGATGGTGCGCTCCTGGACGTAGCCTTCGGGCATG |
| | GCGGACTTGAAGAAGTCGTGCTGCTTCATGTGGTCGGGGTAGCGGCTGAAGCACTGC |
| | ACGCCGTAGGTCAGGGTGGTCACGAGGGTGGGCCAGGGCACGGGCAGCTTGCCGGT |
| | GGTGCAGATGAACTTCAGGGTCAGCTTGCCGTAGGTGGCATCGCCCTCGCCCTCGCC |
| | GGACACGCTGAACTTGTGGCCGTTTACGTCGCCGTCCAGCTCGACCAGGATGGGCAC |
| | CACCCCGGTGAACAGCTCCTCGCCCTTGCTCACCAT |
| 10 | SV40 polyA sequence: |
| | AACTTGTTTATTGCAGCTTATAATGGTTACAAATAAAGCAATAGCATCACAAATTTCAC |
| | AAATAAAGCATTTTTTTCACTGCATTCTAGTTGTGGTTTGTCCAAACTCATCAATGTATC |
| | CCTAGG |
| 11 | SFFV promoter: |
| | CGACTCAGTCTGTCGGAGGACTGGCGCGCCGAGTGAGGGGTTGTGAGCTCTTTTATA |
| | GAGCTCGGGAAGCAGAAGCGCGCGAACAGAAGCGAGAAGCAGGCTGATTGGTTAAT |
| | TCAAATAAGGCGCAGGGTCATTTCAGGTCCTTGGGGGAGCCTGGAAACATCTGATGG |
| | GTCTTAAGAAACTGCTGAGGGTTGGGCCATATCTGGGGACCATCTGTTCTTGGCCTCG |
| | GGCCGGGGCCGAAACTGCGGTGACCATCTGTTCTTGGCCCCGGGCCGGGGCCGAAA |
| | CTGCTCACCGCAGATATCCTGTTTGGCCCAACGTTAGCTATTTTCATGTACCCGCCCTT |
| | GATCTGAACTTCTCTATTCTTGGTTTGGTATTTTTCCATGCCTTGCAAAATGGCGTTAC |
| 12 | P40 promoter: |
| | AAGGTCACCAAGCAGGAAGTCAAAGACTTTTTCCGGTGGGCAAAGGATCACGTGGTT |
| | GAGGTGGAGCATGAATTCTACGTCAAAAAGGGTGGAGCCAAGAAAAGACCCGCCCCC |
| | AGTGACGCAGATATAAGTGAGCCCAAACGGGTGCGCGAGTCAGTTGCGCAGCCATCG |
| | ACGTCAGACGCGGAAGCTTCGATCAACTACGCAGACAGGTACCAAAACAAATGTTCTC |
| | GTCACGTGGGCATGAATCTGATGCTGTTTCCCTGCAGACAATGCGAGAGAATGAATCA |
| | GAATTCAAATATCTGCTTCACTCACGGACAGAAAGACTGTTTAGAGTGCTTTCCCGTGT |
| | CAGAATCTCAACCCGTTTCTGTCGTCAAAAAGGCGTATCAGAAACTGTGCTACATTCAT |
| | CATATCATGGGAAAGGTGCCAGACGCTTGCACTGCCTGCGATCTGGTCAATGTGGATT |
| | TGGATGACTGCATCTTTGAACAATAA |
| 13 | SV40 intron: |
| | GAATTCTCTAGATCAGTTCCAAAGGTTGGAATCTAAAAGAGAGAAACAATTAGAATCA |
| | GTAGTTTAACACATTATACACTTAAAAATTTTATATTTACCTTAGA |
| 14 | TRAC gRNA: |
| | A*G*A*GUCUCUCAGCUGGUACAGUUUUAGAGCUAGAAAUAGCAAGUUAAAAUAAG |
| | GCUAGUCCGUUAUCAACUUGAAAAAGUGGCACCGAGUCGGUGCU* U* U* U; |
| | where the asterisk (*) represents 2′-O-methyl 3′ phosphorothioate |
| 15 | AAV2/AAV6 peptide fwd primer: |
| | CTAACCCTGTGGCCACGG |
| 16 | AAV2/AAV6 peptide rev primer: |
| | CGTCTCTGTCTTGCCACACC |
| 17 | AAV4/AAV6 shuffled fwd primer: |
| | ATTGGCTCGAGGACAACCTCTC |
| 18 | AAV4/AAV6 shuffled rev primer 1: |
| | TTAAACGGTTTATTGATTAACAGGCAATTACAGGG |
| 19 | AAV4/AAV6 shuffled rev primer 2: |
| | TTAAACGGTTTATTGATTAACAAGCAATTACAGGTG |
| 20 | TRAC genomic fwd primer: |
| | GCCAGAGTTATATTGCTGGGGTTTTG |
| 21 | BTK gRNA: |
| | 5′-G*A*U* |
| | GCUCUCCAGAAUCACUGGUUUUAGAGCUAGAAAUAGCAAGUUAAAAUAAGGCUAGU |
| | CCGUUAUCAACUUGAAAAAGUGGCACCGAGUCGGUGCU*U*U*U-3′; where the |
| | asterisk (*) represents 2′-O-methyl 3′ phosphorothioate |
| 22 | BTK genomic forward primer |
| | GGAGTATTGTAGGTTTGGGGAGGC |
| 23 | B-LHA_F |
| | TATCTACTAGTCAGAGGTGGCATCTATATCAGTAAGAC |
| 24 | B-LHA_R |
| | TATGACGTACGGCTTCTTCTTTCTGGAGTTCACC |
| 25 | B-RHA_F |
| | TAGTATGCATTTTCTGAAGCGATCCCAACAG |
| 26 | B-RHA_R |
| | TAGTGGCCGGCCGAGGGATTAAGCAACCAGATGATTAG |

1. A method for identifying an AAV cap gene from an AAV cap library, comprising the following steps:
a) transducing host cells with a test library of replication-incompetent AAV, wherein the replication-incompetent AAV comprise an AAV genome comprising two AAV ITRs flanking a left homology arm and a right homology arm, between which is an AAV cap gene from the AAV cap library, and wherein the sequence of the left homology arm and the sequence of the right homology arm are homologous to sequences at a locus in the genomic DNA of a host cell;
b) isolating genomic DNA from the one or more host cells from step a);
c) detecting integration of the cap gene into the host cell genomic DNA at a position in the locus; and
d) recovering the cap gene from the locus in the genomic DNA, thereby identifying an AAV cap gene, wherein the cap gene is suitable for the production of an AAV vector that can facilitate homologous recombination-mediated gene editing.

2-32. (canceled)


SEQUENCE LISTING (26 sequences)

SEQ ID NO: 1 - DNA, 31 bp, synthetic construct
  source 1..31: mol_type = other DNA; note = VG1228_p40-AAVintron_F
atgcctcgtacgacgtagtccctcgcatcgc

SEQ ID NO: 2 - DNA, 26 bp, synthetic construct
  source 1..26: mol_type = other DNA; note = VG1229_p40-AAVintron_R
atcggcagccatacctgatttaaatc

SEQ ID NO: 3 - DNA, 30 bp, synthetic construct
  source 1..30: mol_type = other DNA; note = VG1234_left-HA-TRAC-F
attgactagtgccgccatggcggccgcggg

SEQ ID NO: 4 - DNA, 31 bp, synthetic construct
  source 1..31: mol_type = other DNA; note = VG1235_left-HA-TRAC-R:
agtcgtcgtacggccgaattcagggtcaggg

SEQ ID NO: 5 - DNA, 32 bp, synthetic construct
  source 1..32: mol_type = other DNA; note = VG1236_NsiIRHA-TRAC_F:
tagtatgcatctggggagcaatcactagccag

SEQ ID NO: 6 - DNA, 34 bp, synthetic construct
  source 1..34: mol_type = other DNA; note = VG1237_FseIRHA-TRAC-R
tatcgggccggccttggccattcctgaagcaagg

SEQ ID NO: 7 - DNA, 145 bp, Adeno-associated dependoparvovirus A
  source 1..145: mol_type = genomic DNA; note = AAV2 ITR left
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 8 - DNA, 145 bp, Adeno-associated dependoparvovirus A
  source 1..145: mol_type = genomic DNA; note = AAV2 ITR right
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 9 - DNA, 720 bp, synthetic construct
  source 1..720: mol_type = other DNA; note = eGFP
ttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccat

SEQ ID NO: 10 - DNA, 125 bp, Macaca mulatta polyomavirus 1
  source 1..125: mol_type = genomic DNA; note = SV40 polyA sequence
aacttgtttattgcagcttataatggttacaaataaagcaatagcatcacaaatttcacaaataaagcatttttttcactgcattctagttgtggtttgtccaaactcatcaatgtatccctagg

SEQ ID NO: 11 - DNA, 402 bp, Spleen Focus-Forming Virus
  source 1..402: mol_type = genomic DNA; note = SSFV promoter
cgactcagtctgtcggaggactggcgcgccgagtgaggggttgtgagctcttttatagagctcgggaagcagaagcgcgcgaacagaagcgagaagcaggctgattggttaattcaaataaggcgcagggtcatttcaggtccttgggggagcctggaaacatctgatgggtcttaagaaactgctgagggttgggccatatctggggaccatctgttcttggcctcgggccggggccgaaactgcggtgaccatctgttcttggccccgggccggggccgaaactgctcaccgcagatatcctgtttggcccaacgttagctattttcatgtacccgcccttgatctgaacttctctattcttggtttggtatttttccatgccttgcaaaatggcgttac

SEQ ID NO: 12 - DNA, 489 bp, Adeno-associated dependoparvovirus A
  source 1..489: mol_type = genomic DNA; note = P40 promoter
aaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 13 - DNA, 104 bp, Macaca mulatta polyomavirus 1
  source 1..104: mol_type = genomic DNA; note = SV40 intron
gaattctctagatcagttccaaaggttggaatctaaaagagagaaacaattagaatcagtagtttaacacattatacacttaaaaattttatatttaccttaga

SEQ ID NO: 14 - RNA, 100 nt, synthetic construct
  source 1..100: mol_type = other RNA
  ncRNA 1..100: ncRNA_class = guide_RNA; note = TRAC gRNA
  modified_base 1: mod_base = OTHER; note = 2' -O-methyl 3' phosphorothioate
  modified_base 2: mod_base = OTHER; note = 2' -O-methyl 3' phosphorothioate
  modified_base 3: mod_base = OTHER; note = 2' -O-methyl 3' phosphorothioate
  modified_base 97: mod_base = OTHER; note = 2' -O-methyl 3' phosphorothioate
  modified_base 98: mod_base = OTHER; note = 2' -O-methyl 3' phosphorothioate
  modified_base 99: mod_base = OTHER; note = 2' -O-methyl 3' phosphorothioate
agagtctctcagctggtacagttttagagctagaaatagcaagttaaaataaggctagtccgttatcaacttgaaaaagtggcaccgagtcggtgctttt

SEQ ID NO: 15 - DNA, 18 bp, synthetic construct
  source 1..18: mol_type = other DNA; note = AAV2/AAV6 peptide fwd primer
ctaaccctgtggccacgg

SEQ ID NO: 16 - DNA, 20 bp, synthetic construct
  source 1..20: mol_type = other DNA; note = AAV2/AAV6 peptide rev primer
cgtctctgtcttgccacacc

SEQ ID NO: 17 - DNA, 22 bp, synthetic construct
  source 1..22: mol_type = other DNA; note = AAV4/AAV6 shuffled fwd primer
attggctcgaggacaacctctc

SEQ ID NO: 18 - DNA, 35 bp, synthetic construct
  source 1..35: mol_type = other DNA; note = AAV4/AAV6 shuffled rev primer 1
ttaaacggtttattgattaacaggcaattacaggg

SEQ ID NO: 19 - DNA, 36 bp, synthetic construct
  source 1..36: mol_type = other DNA; note = AAV4/AAV6 shuffled rev primer 2
ttaaacggtttattgattaacaagcaattacaggtg

SEQ ID NO: 20 - DNA, 26 bp, synthetic construct
  source 1..26: mol_type = other DNA; note = TRAC genomic fwd primer
gccagagttatattgctggggttttg

SEQ ID NO: 21 - RNA, 100 nt, synthetic construct
  source 1..100: mol_type = other RNA
  ncRNA 1..100: ncRNA_class = guide_RNA; note = BTK gRNA
  modified_base 1: mod_base = OTHER; note = 2' -O-methyl 3' phosphorothioate
  modified_base 2: mod_base = OTHER; note = 2' -O-methyl 3' phosphorothioate
  modified_base 3: mod_base = OTHER; note = 2' -O-methyl 3' phosphorothioate
  modified_base 97: mod_base = OTHER; note = 2' -O-methyl 3' phosphorothioate
  modified_base 98: mod_base = OTHER; note = 2' -O-methyl 3' phosphorothioate
  modified_base 99: mod_base = OTHER; note = 2' -O-methyl 3' phosphorothioate
gatgctctccagaatcactggttttagagctagaaatagcaagttaaaataaggctagtccgttatcaacttgaaaaagtggcaccgagtcggtgctttt

SEQ ID NO: 22 - DNA, 24 bp, synthetic construct
  source 1..24: mol_type = other DNA; note = BTK genomic forward primer
ggagtattgtaggtttggggaggc

SEQ ID NO: 23 - DNA, 38 bp, synthetic construct
  source 1..38: mol_type = other DNA; note = B-LHA_F
tatctactagtcagaggtggcatctatatcagtaagac

SEQ ID NO: 24 - DNA, 34 bp, synthetic construct
  source 1..34: mol_type = other DNA; note = B-LHA_R
tatgacgtacggcttcttctttctggagttcacc

SEQ ID NO: 25 - DNA, 31 bp, synthetic construct
  source 1..31: mol_type = other DNA; note = B-RHA_F
tagtatgcattttctgaagcgatcccaacag

SEQ ID NO: 26 - DNA, 38 bp, synthetic construct
  source 1..38: mol_type = other DNA; note = B-RHA_R
tagtggccggccgagggattaagcaaccagatgattag